(19) United States
(12) Patent Application Publication
(10) Pub. No.: US 2023/0101768 A1
(43) Pub. Date: Mar. 30, 2023

(54) METHOD TO TREAT MANGANESE TOXICITY AND MANGANESE-INDUCED PARKINSONISM IN HUMANS

(71) Applicants: THE BOARD OF REGENTS OF THE UNIVERSITY OF TEXAS SYSTEM, Austin, TX (US); THE REGENTS OF THE UNIVERSITY OF CALIFORNIA, Oakland, CA (US)

(72) Inventors: Somshuvra Mukhopadhyay, Austin, TX (US); Chunyi Liu, Austin, TX (US); Donald Smith, Santa Cruz, CA (US)

(21) Appl. No.: 17/819,620

(22) Filed: Aug. 13, 2022

Related U.S. Application Data
(60) Provisional application No. 63/233,026, filed on Aug. 13, 2021.

(51) Int. Cl. A61K 31/472; A61K 31/513; A61K 31/4704; A61K 31/437; A61K 31/5377; A61K 31/4418; A61P 3/12

(52) U.S. Cl. CPC A61K 31/472; A61K 31/513; A61K 31/4704; A61K 31/437; A61K 31/5377; A61K 31/4418; A61P 3/12

(57) ABSTRACT

Disclosed is a method for treating manganese (manganese) toxicity in a subject. The method includes administering to the subject an effective amount of a hypoxia inducible factor (HIF) prolyl hydroxylase inhibitor or a pharmaceutically acceptable salt or solvate thereof.

The present application claims the benefit of priority to U.S. Provisional Patent Application No. 63/233,026, filed Aug. 13, 2021, the entire contents of which is hereby incorporated by reference in its entirety.

### STATEMENT REGARDING FEDERALLY SPONSORED RESEARCH OR DEVELOPMENT

This invention was made with government support Grant no. R01 ES024812 and R01 ES031574 awarded by the National Institutes of Health. The government has certain rights in the invention.

### BACKGROUND OF THE INVENTION

### I. Field of the Invention

The invention generally concerns composition and methods for treating manganese (manganese) toxicity.

### II. Background

Levels of essential metals (e.g., iron (Fe), copper (Cu), zinc (Zn), manganese (Mn) etc.) must be maintained within a narrow physiological range in cells and organisms to avoid deficiency or toxicity. For several essential metals, there are known sophisticated homeostatic pathways that respond to changes in metal levels and adjust metal influx or efflux in a cell- or tissue-specific manner. For example, production of the liver hormone hepcidin increases during iron overload, which induces degradation of the iron efflux transporter ferroportin (Fpn) in iron-exporting cells (e.g., enterocytes, macrophages etc.) to inhibit further release of iron into plasma (1), and the copper efflux transporter ATP7B relocalizes from the trans Golgi network to cytoplasmic vesicles and the apical plasma membrane of hepatocytes to increase copper excretion during excess (2). Dysfunctions in the pathways that regulate metal homeostasis often cause disease. Hepcidin deficiency causes hereditary hemochromatosis characterized by elevated iron levels in the body (1), and ATP7B mutants that fail to traffic in response to copper are associated with Wilson's disease and copper toxicity (3, 4). Further, insights obtained from studies on metal homeostasis have promoted drug discovery—e.g., drugs that target the hepcidin-Fpn response are in clinical trials for treatment of iron metabolism diseases (5). Overall, elucidating the homeostatic control mechanisms that modulate levels of essential metals in human-relevant model systems is essential to understand the pathobiology of, and develop treatments for, metal-induced diseases.

Unlike other essential metals, regulation of manganese homeostasis is poorly understood. The only well-characterized molecular response to manganese in eukaryotes comes from studies in yeast—in this system, the manganese importers Smf1p and Smf2p are stabilized during manganese starvation and degraded under replete or toxic manganese levels (6, 7). However, protein levels of DMT1, the human homolog of yeast Smf1p/Smf2p, have not been reported to respond to changes in manganese levels. In mammalian systems, understanding of manganese homeostasis is limited to insights obtained from elegant radiotracer manganese elimination and tissue distribution studies in rodents performed by Cotzias and coworkers in the 1950s and 1960s, which suggested that changes in manganese excretion are the primary means to control body manganese levels (8-10). But, although five decades have passed since these studies, the underlying molecular mechanisms remain unidentified.

Determining the homeostatic control mechanisms of manganese is biomedically important because of the toxicity of manganese in humans. Briefly, at elevated levels, manganese accumulates in the brain, primarily in the basal ganglia, and induces severe, neurotoxicity that manifests as a parkinsonian-like movement disorder in adults and neuromotor and executive function deficits in children (reviewed in (11)). Established causes of manganese neurotoxicity include elevated exposure from occupational sources in adults (e.g., welding, manufacture of batteries and steel etc.) (11-13) or environmental sources in children and adolescents (e.g., drinking water) (14-24), defective excretion due to chronic liver disease (e.g. alcoholic cirrhosis) (25-31) because manganese is primarily excreted by the liver (10, 32-35), or, as described below, homozygous loss of function mutations in SLC30A10 or SLC39A14 (36-40). Manganese neurotoxicity due to elevated exposure or liver dysfunction is associated with modest ˜1-5 fold or ˜1-7 fold increases in brain manganese levels respectively (11), implying that manganese rapidly becomes toxic as levels exceed the physiologic range (larger increases in tissue manganese levels occur in patients with SLC30A10 or SLC39A14 mutations). Furthermore, while manganese-induced parkinsonism is pathologically distinct from Parkinson's disease (41, 42), elevated manganese induces α-synuclein aggregation (43) and may enhance the risk of developing Parkinson's disease (44). In sum, manganese toxicity and neurotoxicity is an important public health problem, and currently, definitive treatments are not available for manganese toxicity.

### SUMMARY OF THE INVENTION

A discovery has been made that provides a solution to at least one of the aforementioned problems associated with treating manganese toxicity. In one aspect, the solution includes administering to a subject in need thereof, an effective amount of a hypoxia inducible factor (HIF) prolyl hydroxylase inhibitor and/or a pharmaceutically acceptable salt and/or solvate thereof. As shown in Example 1, the Inventors have found that upregulation of SLC30A10 is a homeostatic protective response against manganese toxicity; transcriptional activities of hypoxia inducible factors (HIF), such as HIF1 or HIF2 can upregulate of SLC30A10 and increase SLC30A10 expression; and inhibition of the prolyl hydroxylation of HIFα subunit can stabilize HIF proteins. As shown in Example 2 in a non-limiting manner, it was further found that the small molecule HIF prolyl hydroxylase inhibitors protects against manganese toxicity. Without wishing to be limited by theory, it is believed that HIF prolyl hydroxylase inhibitors can stabilize and/or activate hypoxia inducible factor(s), which can upregulate SLC30A10 gene, which in turn can increase manganese excretion and reduce cellular and organismal manganese levels, thereby protecting against manganese toxicity.

Certain aspects are directed to a method for treating manganese (Mn) toxicity in a subject. The method can include administering to the subject an effective amount of a hypoxia inducible factor (HIF) prolyl hydroxylase inhibitor and/or a pharmaceutically acceptable salt and/or solvate thereof. The HIF prolyl hydroxylase inhibitor can be daprodustat, desidustat, enarodustat, molidustat, roxadustat, vadadustat, or any combinations thereof. In some aspects, the HIF prolyl hydroxylase inhibitor is molidustat, roxadustat, vadadustat, or any combinations thereof. In some particular aspects, the HIF prolyl hydroxylase inhibitor is daprodustat. In some particular aspects, the HIF prolyl hydroxylase inhibitor is desidustat. In some particular aspects, the HIF prolyl hydroxylase inhibitor is enarodustat. In some particular aspects, the HIF prolyl hydroxylase inhibitor is molidustat. In some particular aspects, the HIF prolyl hydroxylase inhibitor is roxadustat. In some particular aspects, the HIF prolyl hydroxylase inhibitor is vadadustat. In some aspects, the subject has manganese-induced neurotoxicity. In some aspects, the subject is at a risk of developing manganese-induced neurotoxicity. In some aspects, the method can be used to treat manganese-induced neurotoxicity. In some aspects, the subject has manganese-induced parkinsonism. In some aspects, the subject is at a risk of developing manganese-induced parkinsonism. In some aspects, the method can be used to treat manganese-induced parkinsonism. In certain aspects, the subject has cirrhosis. In certain aspects, the subject has an increased manganese concentration in one or more tissues and/or organs when compared to a standard. The one or more tissues and/or organs can include but not limited to blood, brain, liver, bone, and intestine. In certain aspects, the subject has an increased blood manganese concentration when compared to a standard. In certain aspects, the subject has a blood manganese concentration greater than 7 μg/L. In some aspects, the subject has an increased bone manganese concentration when compared to a standard. In some aspects, the subject has an increased liver manganese concentration when compared to a standard. In some aspects, the subject has an increased brain manganese concentration when compared to a standard. In some aspects, the subject has an increased intestine manganese concentration when compared to a standard. For the respective organs, the standard can be a known standard, or previous standard established for the subject. In some aspects, the subject has manganese deposition in the liver and/or brain. In some aspects, manganese concentration in the basal ganglia of the subject is higher, compared to manganese concentration in other areas of the brain. In some aspects, the subject has neuronal degeneration at the basal ganglia, or is at a risk of developing neuronal degeneration at the basal ganglia. In some aspects, the subject has homozygous loss of function mutations in the SLC30A10 gene and/or the SLC39A14 gene. In some aspects, the subject has single nucleotide polymorphism in the SLC30A10 gene. In some aspects, the subject has SLC39A14 deficiency in the brain, liver, and/or intestine. In some aspects, the subject has increased expression of SLC30A10 in the brain, liver, and/or intestine. The HIF prolyl hydroxylase inhibitor or a pharmaceutically acceptable salt or solvate thereof can be administered orally, intravenously, parenterally, subcutaneously, or intramuscularly. The subject can be human.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients, compositions, steps, etc disclosed throughout the specification. With respect to the transitional phrase “consisting essentially of,” in one non-limiting aspect, a basic and novel characteristic of the compositions and methods of the present invention are their abilities treat manganese toxicity.

The terms “treat,” “treating” or “treatment” refer to both therapeutic treatment and prophylactic or preventive measures, wherein the object is to either induce or prevent or slow down (lessen) an undesired physiological change. For purposes of this invention, beneficial or desired results include, but are not limited to, alleviation of one or more symptoms of disease (e.g. manganese-toxicity, manganese induced neurotoxicity, and/or manganese induced parkinsonism), diminishment of extent of disease, stabilized (i.e., not worsening) state of disease, delay or slowing of disease progression, amelioration of the disease state, and remission (whether partial or total), whether detectable or undetectable.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F: (A): manganese exposure increases SLC30A10 expression in the liver of 129S4/SvJaeJ and intestines of C57BL/6J mice. qRT-PCR analyses from indicated tissues of 129S4/SvJaeJ or C57BL/6J mice treated with vehicle or oral manganese (˜15 mg absolute manganese/kg body weight daily) from PND1 until 8 weeks of age. Animals were euthanized at 8 weeks of age. For each strain, mean expression in vehicle treated mice was normalized to 1. N=10 vehicle and 12 manganese for 129S4/SvJaeJ, and 7 vehicle and 8 manganese for C57BL/6J mice. Mean±SE. *P<0.05 by two-way ANOVA (strain and manganese treatment as independent variables) and Sidak's post hoc test for indicated comparisons; n.s., not significant. (B)-(F): manganese-induced changes in expression of manganese transporters in 12954/SvJaeJ or C57BL/6J mice. Samples analyzed for SLC30A10 expression in (A) were processed for analyses of transporters indicated above by qRT-PCR. Sample processing, animal number, and normalization were identical to (A). Mean±SE. *P<0.05 by two-way ANOVA (strain and manganese treatment as independent variables) and Sidak's post hoc test for indicated comparisons; n.s., not significant.

FIG. 2A-D: manganese upregulates SLC30A10 expression in HepG2 cells. (A) and (C): qRT-PCR analyses. In (A), manganese treatment was for 4 h at indicated concentrations. In (C), manganese was used at 250 μM for indicated times. Expression without manganese treatment is normalized to 1. N=3 for (A) and (C). Mean±SE. *P<0.05 by one-way ANOVA and Dunnett's post hoc test for the comparison between no manganese and other conditions. (B): Intracellular manganese levels after 4 h exposure to manganese. N=4. Mean±SE. *P<0.05 by one-way ANOVA and Dunnett's post hoc test for the comparison between no manganese and other conditions. (D): Immunoblot assays after treatment with 250 μM manganese for indicated times.

FIG. 3A-G: The manganese-induced upregulation of SLC30A10 in HepG2 cells requires a hypoxia response element in the SLC30A10 promoter. (A): qRT-PCR analyses after pre-treatment of cells with vehicle DMSO or actonimycin D (5 μM) for 30 min followed by exposure to 0 or 250 μM manganese for 4 h. Expression without manganese treatment was normalized to 1 in DMSO or actinomycin D treated cells separately. N=3. Mean±SE. *P<0.05 by t-test. (B) and (F): Schematic of luciferase (Luc) reporter constructs used (B) and the required hypoxia response element (shown in red) in the SLC30A10 promoter (F). Numbers indicate position relative to the start of transcription. (C) and (G): Relative normalized luciferase signal after manganese treatment in cells expressing indicated luciferase constructs. The hypoxia response element (HRE) highlighted in red in (F) was deleted in the ΔHRE construct in (G). Cells were treated with or without 250 μM manganese for 16 h. Luciferase signals were normalized to protein in each sample. For each construct, values after manganese treatment are expressed relative to that without manganese normalized to 1. N≥3. Mean±SE. *P<0.05 by one-way ANOVA and Dunnett's post hoc test for the comparison between luciferase only and other conditions in (C) and t-test in (G). (D) and (E): c-Jun, C/EBPB, YY1, P53, and RXRα are not required for the manganese-induced increase in SLC30A10 expression. (D), qRT-PCR analyses in HepG2 cells stably expressing dominant negative (DN) versions of c-Jun, C/EBPB, YY1, P53, or RXRα, or infection control cells treated with 0 or 250 μM manganese for 4 h. Expression without manganese treatment was normalized to 1 for each infection condition. N=3-4. There were no differences between groups by t-test. (E), qRT-PCR analyses in HepG2 cells infected with indicated dominant negative mutants or infection control cells. Expression in infection control cells was normalized to 1. N=3. *P<0.05 by t-test.

FIG. 4A-D: manganese stabilizes and induces nuclear accumulation of HIF1α and HIF2α protein in HepG2 cells. (A) and (D): Immunoblot (A) and immunofluorescence (D) analyses after treatment with 0 or 250 μM manganese for indicated times (A) or 4 h (D). (B) and (C): Quantification of HIF1α or HIF2α levels normalized to tubulin from (A). N=3. Mean±SE. *P<0.05 by one-way ANOVA and Dunnett's post hoc test for comparisons between 0 h and other groups.

FIG. 5A-G: HIF1 or HIF2 are required and sufficient for the manganese-induced upregulation of SLC30A10 expression. (A)-(D): qRT-PCR analyses in HepG2 cells stably expressing shRNAs with a scrambled sequence or targeting HIF1α (A), HIF2α (B), HIF1β/ARNT (C) or HIF1α and HIF2α (D), and treated with or without 250 μM manganese for 4 h. Scramble shRNA sequence 1 was used for experiments with HIF1α shRNA (A) while scramble shRNA sequence 2 was used for the other experiments (B)-(D). For each infection condition, expression without manganese was normalized to 1. N=3-4. Mean±SE. *P<0.05 by t-test (A), (B), and (D) or one-way ANOVA and Dunnett's post hoc test for the comparison between scramble shRNA and other groups (C). (E): qRT-PCR in HepG2 cells treated with or without 10 μM LW6 for 1 h followed by exposure to 0 or 250 μM manganese for 4 h. For cells that did or did not receive LW6, expression without manganese was separately normalized to 1. N=5. Mean±SE. *P<0.05 by t-test. (F): Relative normalized luciferase activity in cells stably expressing SLC30A10^{-368 to -210}luc after treatment with or without 10 μM LW6 for 1 h followed by 0 or 250 μM manganese for 16 h. For cells that did or did not receive LW6, separately, luciferase activity was measured with or without manganese treatment, normalized to protein concentration, and the value for the group without manganese treatment was expressed as 1. N=3. Mean±SE. *P<0.05 by t-test. (G): qRT-PCR analyses in control HepG2 cells or cells stably expressing VHL-insensitive mouse HIF1α or HIF2α. Expression in control infected cells was normalized to 1. N=3. Mean±SE. *P<0.05 by one-way ANOVA and Dunnett's post hoc test for comparisons between control and other groups.

FIG. 6A-B: manganese inhibits the prolyl hydroxylation of HIF1α. (A): qRT-PCR analyses in HepG2 cells treated with 250 μM manganese for indicated times. Expression in cells that did not receive manganese was normalized to 1. N=3. Mean±SE. *P<0.05 by one-way ANOVA and Dunnett's post hoc test for comparison between no manganese and other groups. (B): Immunoblot analyses of whole-cell lysates obtained from HepG2 cells treated with or without 10 μM MG132 for 1 h followed by exposure to 0 or 250 μM manganese for 4 h. HIF1α-OH, prolyl hydroxylated HIF1α.

FIG. 7A-C: The activation of HIF transcription factors by manganese is a homeostatic protective response against manganese toxicity. (A): ICP-MS analyses to measure intracellular metal levels in HepG2 cells stably infected with scramble shRNA (sequence 2) or HIF1β/ARNT shRNA (sequence 1) and treated with or without 125 μM manganese for 16 h. N=3. Mean±SE. *P<0.05 by two-way ANOVA (shRNA and manganese treatment as independent variables) and Sidak's post hoc test for indicated comparisons; n.s., not significant. (B): Intracellular levels of Fe, Cu, and Zn are not affected by knockdown of HIF1β/ARNT. Amounts of Fe, Cu, and Zn were quantified in samples used for manganese measurements in (A). Sample size is identical to (A). Data are mean±SE. There were no differences between groups using two-way ANOVA. (C): Cell viability assays in scramble (sequence 2) or HIF1β/ARNT shRNA (sequence 1)-infected HepG2 cells treated with 0, 0.5, or 1 mM manganese for 16 h. Viability of scramble or ARNT shRNA-infected cells that did not receive manganese was separately normalized to 100. N=3. Mean±SE. *P<0.05 by two-way ANOVA (shRNA and manganese treatment as independent variables) and Sidak's post hoc test for comparisons between infection conditions at each manganese dose.

FIG. 8A-F: Prolyl hydroxylase inhibitors enhance SLC30A10 levels, reduce intracellular manganese, and protect HepG2 cells against manganese toxicity. (A): Immunoblot assays in cells treated with or without 20 μM roxadustat or 80 μM vadadustat for 16 h. (B) and (C): qRT-PCR after treatment with indicated doses of roxadustat or vadadustat for 16 h (B) or 20 μM roxadustat or 80 μM vadadustat for indicated times (C). Expression in cells that did not receive the drug was normalized to 1. N≥3. Mean±SE. *P<0.05 by one-way ANOVA and Dunnett's post hoc test for comparison between no drug and other conditions. (D): Intracellular manganese levels in cells treated with or without 20 μM roxadustat or 80 μM vadadustat for 16 h followed by exposure to 0 or 125 μM manganese for 24 h. N=5-6. Mean±SE. *P<0.05 by two-way ANOVA (drug and manganese treatment as independent variables) and Tukey Kramer post hoc test for indicated comparisons; n.s., not significant. (E): Effect of roxadustat or vadadustat on intracellular Fe, Cu, and Zn. Fe, Cu, and Zn levels were quantified in samples used for manganese measurements in FIG. 8(D). Sample size is identical to FIG. 8(D). Data are mean±SE. Within cells that did or did not receive manganese, there was no effect of roxadustat or vadadustat treatment on Fe, Cu, or Zn levels by two-way ANOVA. (F): Cell viability assays after treatment with or without 20 μM roxadustat or 80 μM vadadustat for 16 h followed by exposure to indicated levels of manganese for 24 h. Viability of cells treated with or without prolyl hydroxylase inhibitors in the absence of manganese exposure were separately normalized to 100. N=3. Mean±SE. *P<0.05 by two-way ANOVA (drug and manganese treatment as independent variables) and Sidak's post hoc test for comparisons between treatment conditions at each manganese dose.

FIG. 9A-C: Molidustat increases HIF1α and HIF2α protein as well as SLC30A10 expression, and protects against manganese-induced cell death. (A): Immunoblot analyses after treatment of HepG2 cells with 0 or 20 μM molidustat for 16 h. (B): qRT-PCR analyses after treatment of HepG2 cells with indicated levels of molidustat for 16 h. N=3. Mean±SE. *P<0.05 by one-way ANOVA and Dunnett's post hoc test for comparison between no drug and other conditions. (C): Cell viability assays in HepG2 cells after treatment with or without 20 μM molidustat for 16 h followed by exposure to indicated levels of manganese for 24 h. Viability of cells treated with or without molidustat in the absence of manganese exposure were separately normalized to 100. N=4. Mean±SE. *P<0.05 by two-way ANOVA (drug and manganese treatment as independent variables) and Sidak's post hoc test for comparisons between treatment conditions at each manganese dose.

FIG. 10A-B: manganese treatment upregulates SLC30A10 in a HIF-dependent manner in primary human hepatocytes. (A) and (B): qRT-PCR assays in cells treated with or without 10 μM LW6 for 1 h followed by 0 or 250 μM manganese for 4 h (A) or with or without 20 μM roxadustat or 80 μM vadadustat for 16 h (B). For (A), separately for cells that did or did not receive LW6, expression in the absence of manganese treatment was normalized to 1. For (B), expression in the absence of drug treatment was normalized to 1. N=3. Mean±SE. *P<0.05 by t-test (A) or one-way ANOVA and Dunnett's post hoc test for the comparison between no drug and other conditions (B).

FIG. 11A-D: Roxadustat protects mice against manganese neurotoxicity. (A): qRT-PCR assays from indicated tissues of C57BL/6J mice harvested after treatment with vehicle or roxadustat (10 mg/kg body weight daily i.p.) for 4 weeks (starting at ˜4-weeks of age). Mean expression in vehicle treated mice was normalized to 1. N=5/treatment. Mean±SE. *P<0.05 by t-test. (B): Open-field activity data for vertical movement in mice treated with vehicle or roxadustat as described in (A) along with or without exposure to manganese in drinking water (˜30 mg absolute manganese/kg body weight daily). Treatment was at ages and for the duration described in (A). Open-field analysis was performed before euthanasia. N=5/group. Mean±SE. *P<0.05 by repeated measures two-way ANOVA and Tukey Kramer post hoc test for indicated comparisons. (C): ICP-MS metal analyses from indicated tissues of animals used for neurobehavioral analyses in B. N=5/group. Mean±SE. Indicated p values are for two-way ANOVA analyses using roxadustat and manganese as independent variables; n.s., not significant. (D): Effect of roxadustat on blood and brain Fe, Cu, and Zn levels. Fe, Cu, and Zn levels were quantified in tissue samples used for manganese measurements in (C). Sample size is identical to (C). Data are mean±SE. There was no effect of roxadustat on Fe, Cu, or Zn levels in blood or brain by two-way ANOVA.

### DETAILED DESCRIPTION OF THE INVENTION

Altered expression/activity of metal transporters can control metal homeostasis. But, until recently, transporters critical for manganese influx and efflux in humans were unknown, and this gap in knowledge hindered studies on manganese homeostasis. Needed breakthroughs came over the last few years when homozygous loss-of-function mutations in SLC30A10 or SLC39A14 were reported to increase manganese levels in the blood and brain and induce hereditary manganese neurotoxicity in humans (36-40), while homozygous loss-of-function mutations in SLC39A8 were reported to lead to the onset of hereditary manganese deficiency (45, 46). It was discovered that SLC30A10 is a cell-surface localized manganese efflux transporter that transports manganese from the cytosol to the cell exterior, reduces cellular manganese levels, and protects against manganese toxicity (47). The efflux activity of SLC30A10 is specific to manganese (47-49). Using full-body and tissue-specific Slc30a10 knockout mice, two critical functions of SLC30A10 at the whole organism level were identified—SLC30A10 localizes to the apical domain of hepatocytes and enterocytes to mediate hepatic and intestinal manganese excretion, and additionally, SLC30A10 is active in the brain where it mediates neuronal manganese efflux to provide additional neuroprotection (50, 51) (note that ˜80% of the body burden of manganese is excreted by the liver, and the intestines excrete the remaining ˜20% (10, 33)). The role of SLC30A10 in manganese excretion was subsequently also confirmed (52). Manganese neurotoxicity upon loss-of-function of SLC30A10 is a consequence of blocked manganese excretion and an inhibition of neuronal manganese efflux (11, 53). Similar studies on SLC39A14 identified it to be a manganese influx transporter that, at the organism level, is essential for the transport of manganese from blood into the liver and intestines for subsequent excretion by SLC30A10 (50, 54-59). Loss-of-function of SLC39A14 also induces manganese neurotoxicity by blocking manganese excretion (11, 53). Other work identified SLC39A8 to be a hepatic manganese importer that reclaims manganese excreted into bile to prevent manganese deficiency (60). Unlike SLC30A10, SLC39A14 and SLC39A8 also transport other divalent metals in vitro (61), but their primary in vivo function under physiological conditions appears to be manganese transport. Put together, the recent studies establish SLC30A10, SLC39A14, and SLC39A8 as essential human manganese transporters.

Based on the identification of SLC30A10, SLC39A14, and SLC39A8 as manganese transporters, the inventors found that upregulation of SLC30A10 provides a pathway to enhance manganese excretion and reduce manganese levels during manganese toxicity and protects against manganese toxicity. Without wishing to be limited by theory, it is believed that, the underlying mechanism is the activation of hypoxia inducible factor (HIF) 1 and HIF2 due to an inhibitory effect of elevated levels of manganese on the prolyl hydroxylation and subsequent degradation of HIF proteins. Further, it is shown that small molecule inhibitors of prolyl hydroxylase enzymes, e.g., HIF prolyl hydroxylase inhibitor, which stabilize HIF proteins, protect cells and mice against manganese toxicity.

In humans, elevated exposure to manganese can occur via inhalation in occupational settings and/or orally via drinking water due to environmental contamination (11). The oral manganese exposure regimen utilized for the mechanistic studies described herein modeled human environmental manganese exposure (11).

As mentioned, currently definitive treatments are not available for manganese neurotoxicity. Bringing a new drug into clinical use can be challenging, and likelihood of success might be higher if an existing drug can be repurposed. It was discovered that small molecule prolyl hydroxylase inhibitors can protect cells and mice against manganese toxicity. Without wishing to be bound by theory it is believed that these prolyl hydroxylase inhibitors can protect against manganese toxicity by activating HIF-dependent transcription by stabilizing HIFα subunits, and thereby increasing SLC30A10 levels and increasing manganese excretion. In some aspects, these drugs may not need to cross the blood brain barrier for efficacy, which substantially enhances the possibility of therapeutic effectiveness.

Methods for treating manganese (Mn) toxicity in a subject are described. In one aspect, the method includes administering to a subject in need thereof an effective amount of a hypoxia inducible factor (HIF) prolyl hydroxylase inhibitor and/or a pharmaceutically acceptable salt and/or solvate thereof. Mechanistic studies described herein show that: 1) elevated manganese transcriptionally upregulate SLC30A10 gene, a manganese efflux transporter, in the liver and intestines; 2) a hypoxia response element in the SLC30A10 promoter was necessary for the upregulation; 3) the transcriptional activities of hypoxia inducible factor (HIF) 1 or HIF2 were required and sufficient for the SLC30A10 response; and 4) elevated manganese activated HIF1/HIF2 by blocking the prolyl hydroxylation of HIF proteins necessary for their degradation. Further it the shown in a non-limiting manner in Example 2, that prolyl hydroxylase inhibitors that stabilize HIF proteins afforded cellular protection against manganese toxicity, and also ameliorated the in vivo manganese-induced neuromotor deficits in mice.

In some aspects, the HIF prolyl hydroxylase inhibitor can be daprodustat, desidustat, enarodustat, molidustat, roxadustat, vadadustat, or any combinations thereof. In some aspects, the HIF prolyl hydroxylase inhibitor is daprodustat. In some aspects, the HIF prolyl hydroxylase inhibitor is desidustat. In some aspects, the HIF prolyl hydroxylase inhibitor is enarodustat. In some aspects, the HIF prolyl hydroxylase inhibitor is molidustat. In some aspects, the HIF prolyl hydroxylase inhibitor is roxadustat. In some aspects, the HIF prolyl hydroxylase inhibitor is vadadustat. Daprodustat (PubChem CID: 91617630) has the IUPAC name 2-[(1,3-dicyclohexyl-2,4,6-trioxo-1,3-diazinane-5-carbonyl)amino]acetic acid, and chemical formula of Formula I. Desidustat (PubChem CID: 75593290) has the IUPAC name 2-[[1-(cyclopropylmethoxy)-4-hydroxy-2-oxoquinoline-3-carbonyl]amino]acetic acid, and chemical formula of Formula II. Enarodustat (PubChem CID: 50899324) has the IUPAC name 2-[[7-oxo-5-(2-phenylethyl)-3H-[1,2,4]triazolo[1,5-a]pyridine-8-carbonyl]amino]acetic acid, and chemical formula of Formula III. Molidustat (PubChem CID: 59603622) has the IUPAC name 2-(6-morpholin-4-ylpyrimidin-4-yl)-4-(triazol-1-yl)-1H-pyrazol-3-one, and chemical formula of Formula IV. Roxadustat (PubChem CID: 11256664) has the IUPAC name 2-[(4-hydroxy-1-methyl-7-phenoxyisoquinoline-3-carbonyl)amino]acetic acid, and chemical formula of Formula V. Vadadustat (PubChem CID 23634441) has the IUPAC name 2-[[5-(3-chlorophenyl)-3-hydroxypyridine-2-carbonyl]amino]acetic acid and chemical formula of Formula VI.

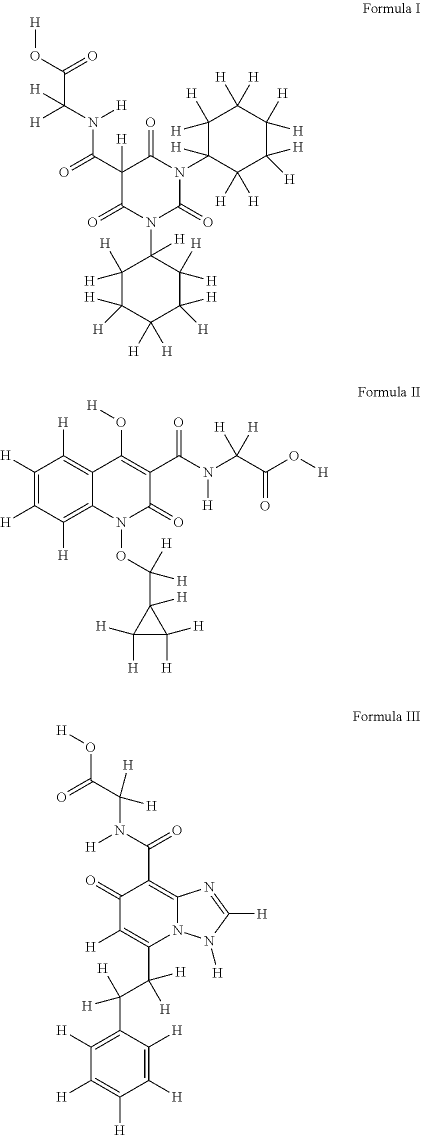 [SMILES: O=C(O)CNC(=O)C1C(=O)N(C2CCCCC2)C(=O)N(C2CCCCC2)C1=O ; O=C(O)CNC(=O)c1c(O)c2ccccc2n(OCC2CC2)c1=O ; O=C(O)CNC(=O)c1c(=O)cc(CCc2ccccc2)n2[nH]cnc12]

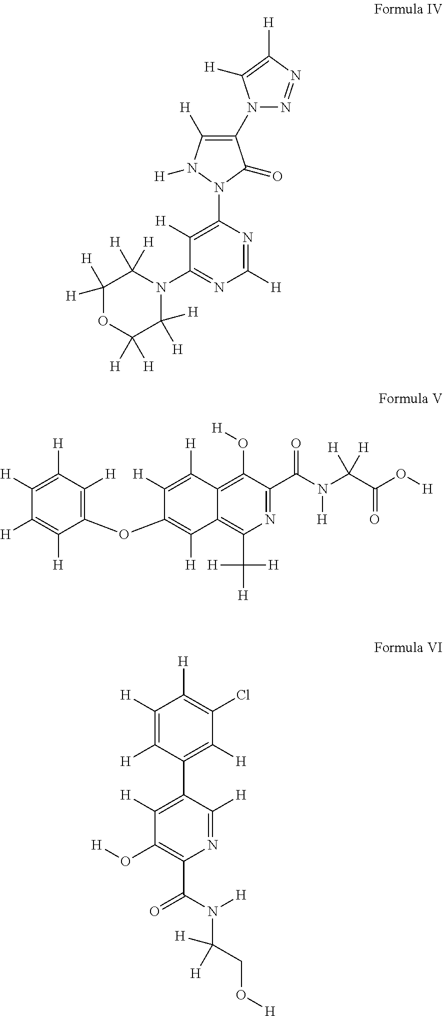 [SMILES: O=c1c(-n2ccnn2)c[nH]n1-c1cc(N2CCOCC2)ncn1 ; Cc1nc(C(=O)NCC(=O)O)c(O)c2ccc(Oc3ccccc3)cc12 ; O=C(NCCO)c1ncc(-c2cccc(Cl)c2)cc1O]

The methods described herein can be used for treating manganese toxicity in a subject. In some aspects, the methods can be used for treating manganese induced neurotoxicity in a subject. In some aspects, the methods can be used for treating manganese-induced parkinsonism in a subject. Certain aspects, are directed to use of the HIF prolyl hydroxylase inhibitor(s) and/or a pharmaceutically acceptable salt(s) and/or solvate(s) thereof for treating manganese toxicity, treating manganese induced neurotoxicity, and/or treating manganese induced parkinsonism in a subject. The HIF prolyl hydroxylase inhibitor(s) can be used to increase manganese excretion in a subject. In certain aspects, the subject has an increased manganese concentration in one or more tissues and/or organs when compared to a standard. For a tissue and/or organ, the standard manganese concentration can be a known standard, or previous standard established for the subject. Depending on the subject, the tissue and/or organ specific standard would be apparent to a clinician and/or a physician. The one or more tissues and/or organs can include but not limited to blood, brain, liver, bone, and intestine. In some aspects, the subject has an increased blood manganese concentration when compared to a standard blood manganese concentration. It is known that blood manganese concentration can vary depending on age, sex, race, and/or genetic factors of a subject, along with study region, time of collection etc. Traditionally 7 to 10 μg/L has been indicated as “normal” blood manganese concentration (Hauser et al, 1994; Sphar et al, 1996; Tuschl et al, 2016), however lower values such as 3 μg/L (Quadri et al, 2012) and higher values such as <17.5 μg/L (Tushcl et al., 2012) have also been reported. It is also known that newborns and young children can have higher blood manganese concentration (up to 35 ug/L or higher). Further, blood manganese concentration can also be naturally elevated in pregnant women across trimesters, even as high as 25 ug/L blood manganese in the third trimester. A subject suffering from manganese induced neurotoxicity might or might not have elevated blood manganese concentration. In some aspects, the blood manganese concentration of the subject can be equal to any one of, greater than any one of, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 and 100 ug/L. In some aspects, the subject has increased bone manganese concentration when compared to a standard bone manganese concentration. A subject suffering from manganese induced neurotoxicity might or might not have elevated bone manganese concentration. In certain aspects, the subject has manganese deposition in one or more tissues and/or organs. The one or more tissues and/or organs can include but not limited to brain, liver, bone, and intestine. In certain aspects, the subject has manganese deposition in liver. In certain aspects, the subject has manganese deposition in intestine. In certain aspects, the subject has manganese deposition in brain. A subject suffering from manganese induced neurotoxicity might have manganese deposition in liver, intestine and/or brain. In certain aspects, manganese concentration in the basal ganglia of the subject is higher, compared to manganese concentration in other areas of the brain of the subject. In certain aspects, the subject has neuronal degeneration at the basal ganglia, or is at a risk of developing neuronal degeneration at the basal ganglia. The subject can have and/or is diagnosed with manganese toxicity. In some aspects, the subject is at a risk of developing manganese toxicity. In some aspects, the subject has and/or has been diagnosed with manganese induced neurotoxicity. In some aspects, the subject is at a risk of developing manganese induced neurotoxicity. In some aspects, the subject has and/or has been diagnosed with manganese-induced parkinsonism. In some aspects, the subject is at a risk of developing manganese-induced parkinsonism. Risk of developing of manganese toxicity, manganese induced neurotoxicity and/or manganese induced parkinsonism in a subject can depend on, for example in a non-limiting manner, on elevated exposure from occupational sources (such as but not limited to welding, manufacture of batteries and steel etc.) or environmental sources (such as but not limited to drinking water); defective excretion due to chronic liver disease (such as but not limited to alcoholic cirrhosis); and/or due to genetic factors such as but not limited to, homozygous loss of function mutations in SLC30A10 or SLC39A14. manganese concentration and deposition in an organ and/or a tissue can be measured by any suitable method, non-limiting examples includes—bone manganese concentration can be determined using neutron activation analyses, and brain and liver manganese deposition can be measured using magnetic resonance imaging (MRI), such as T1-weighted MRI scan as hyperintensity. Manganese toxicity, manganese induced neurotoxicity, and manganese induced parkinsonism can be diagnosed using any suitable method, including but not limited to diagnosis methods that use magnetic resonance imaging (MRI), such as T1-weighted MRI scan as hyperintensity. In some aspects, the subject has homozygous loss of function mutation in the SLC39A14 gene. In some aspects, the subject does not have a mutation in the SLC39A14 gene. In some aspects, the subject does not have a homozygous loss of function mutation in the SLC39A14 gene. In some aspects, the subject has a homozygous loss of function mutation in the SLC30A10 gene. In some aspects, the subject has a single nucleotide polymorphism in the SLC30A10 gene. In some aspects, the subject does not have a mutation in the SLC30A10 gene. In some aspects, the subject does not have a homozygous loss of function mutation in the SLC30A10 gene. In some aspects, the subject does not have a single nucleotide polymorphism in the SLC30A10 gene. In some aspects, the subject has a deficiency of SLC39A14 in the brain, liver, and/or intestine. In some aspects, a majority of cellular SLC30A10 in the brain, liver, and/or intestine of the subject, is localized inside the endoplasmic reticulum. In some aspects, the subject has increased expression of SLC30A10 in the brain, liver, and/or intestine. In some aspects, subjects without homozygous loss of function mutation in the SLC30A10 gene might have increased expression of SLC30A10 in the brain, liver, and/or intestine due to manganese toxicity, however the increased expression (without administration of the HIF prolyl hydroxylase inhibitor compounds) might not be sufficient enough to cure manganese toxicity. In certain aspects, the subject can be human. The subject can be: a child, e.g., can have an age below 11 years; a teenager, e.g., can have an age 11 to 19 years; a young adult, e.g., can have an age above 19 to 30 years; an adult e.g., can have an age above 30 years to 60 years; or an elderly person e.g., can have an age above 60 years.

### A. ADMINISTRATION OF THERAPEUTIC COMPOSITIONS

The therapy provided herein may comprise administration of a HIF prolyl hydroxylase inhibitor and/or pharmaceutically acceptable salt and/or solvate thereof. In certain aspects, one or more HIF prolyl hydroxylase inhibitor(s) are administered. In certain aspects, a combination of HIF prolyl hydroxylase inhibitor (and/or pharmaceutically acceptable salt and/or solvate thereof), such as a first HIF prolyl hydroxylase inhibitor and a second HIF prolyl hydroxylase inhibitor, are administered. The compounds (e.g., HIF prolyl hydroxylase inhibitors) may be administered in any suitable manner known in the art. For example, the first and second HIF prolyl hydroxylase inhibitors may be administered sequentially (at different times) or concurrently (at the same time). In some embodiments, the first and second HIF prolyl hydroxylase inhibitors are administered in a separate composition. In some embodiments, the first and second HIF prolyl hydroxylase inhibitors are in the same composition.

In some embodiments, the first HIF prolyl hydroxylase inhibitor and the second HIF prolyl hydroxylase inhibitor are administered substantially simultaneously. In some embodiments, the first HIF prolyl hydroxylase inhibitor and the second HIF prolyl hydroxylase inhibitor are administered sequentially. In some embodiments, the first HIF prolyl hydroxylase inhibitor, the second HIF prolyl hydroxylase inhibitor, and a third HIF prolyl hydroxylase inhibitor are administered sequentially. In some embodiments, the first HIF prolyl hydroxylase inhibitor is administered before administering the second HIF prolyl hydroxylase inhibitor. In some embodiments, the first HIF prolyl hydroxylase inhibitor is administered after administering the second HIF prolyl hydroxylase inhibitor.

Embodiments of the disclosure relate to compositions and methods comprising one or more HIF prolyl hydroxylase inhibitors. The different HIF prolyl hydroxylase inhibitors, may be administered in one composition or in more than one composition, such as 2 compositions, 3 compositions, or 4 compositions. Various combinations of the agents may be employed.

The therapeutic composition of the disclosure may be administered by the same route of administration or by different routes of administration. In some embodiments, the therapeutic composition is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician. The therapeutic composition can include a HIF prolyl hydroxylase inhibitor, and/or pharmaceutically acceptable salt and/or solvate thereof.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the therapeutic composition containing the HIF prolyl hydroxylase inhibitor and/or pharmaceutically acceptable salt and/or solvate thereof is administered at a dose of between 0.01 mg/kg and 5000 mg/kg (weight of the compound/weight of the subject). In some embodiments, the HIF prolyl hydroxylase inhibitor and/or pharmaceutically acceptable salt and/or solvate thereof is administered at a dose of at least, at most, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900,1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents (e.g., HIF prolyl hydroxylase inhibitor(s) and/or pharmaceutically acceptable salt and/or solvate thereof). Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 mg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

1. Pharmaceutical Compositions

In certain aspects, the compositions or agents for use in the methods, such as HIF prolyl hydroxylase inhibitor compound(s) and/or pharmaceutically acceptable salt and/or solvate thereof, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e., to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

2. Other Agents

It is contemplated that other agents may be used in combination with certain aspects of the present embodiments to improve the therapeutic efficacy of treatment. These additional agents include agents that can reduce manganese toxicity in a subject. The therapeutic composition described herein can be combined with other therapy(ies) for manganese toxicity. For example, a first composition or ingredient is “A” and a second composition or ingredient is “B” can be combined and/or excluded, in a non-limiting manner as follows:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A.

### B. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Upregulation of SLC30A10 is a Critical, Homeostatic Protective Response

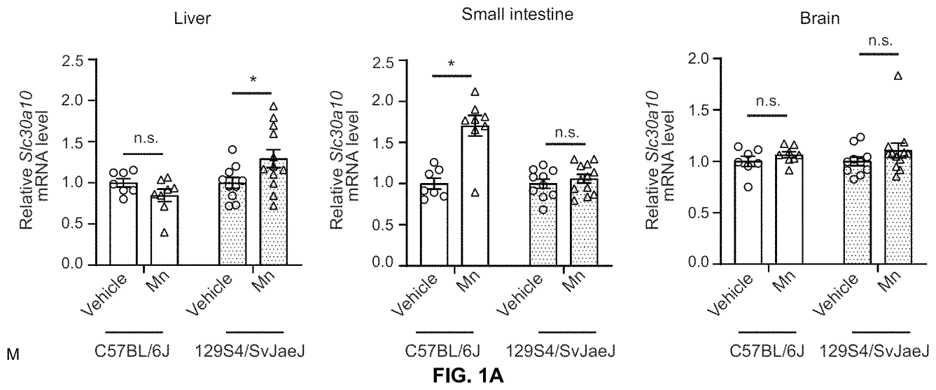

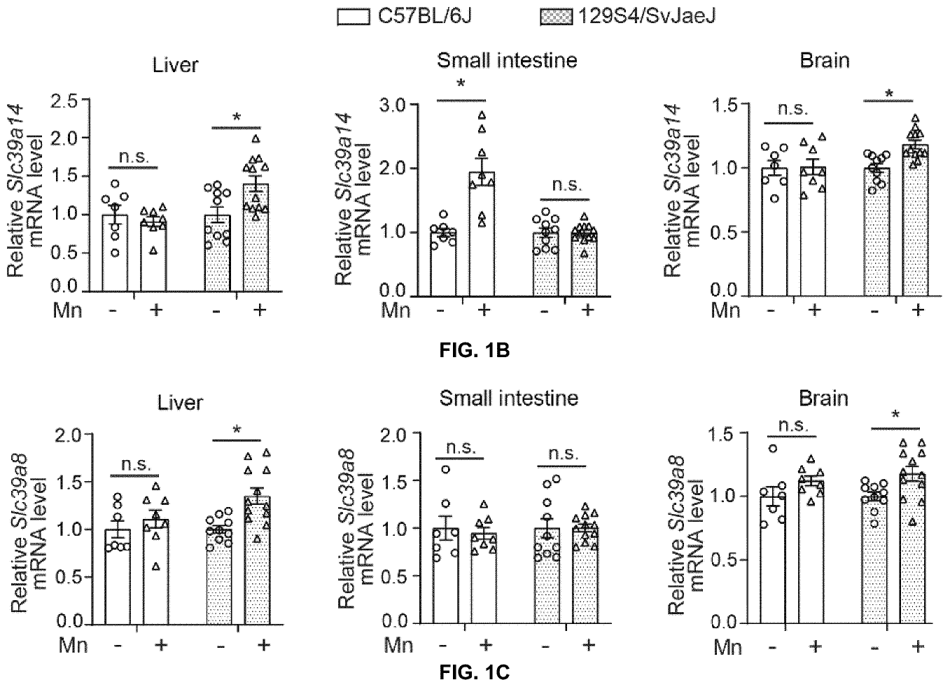

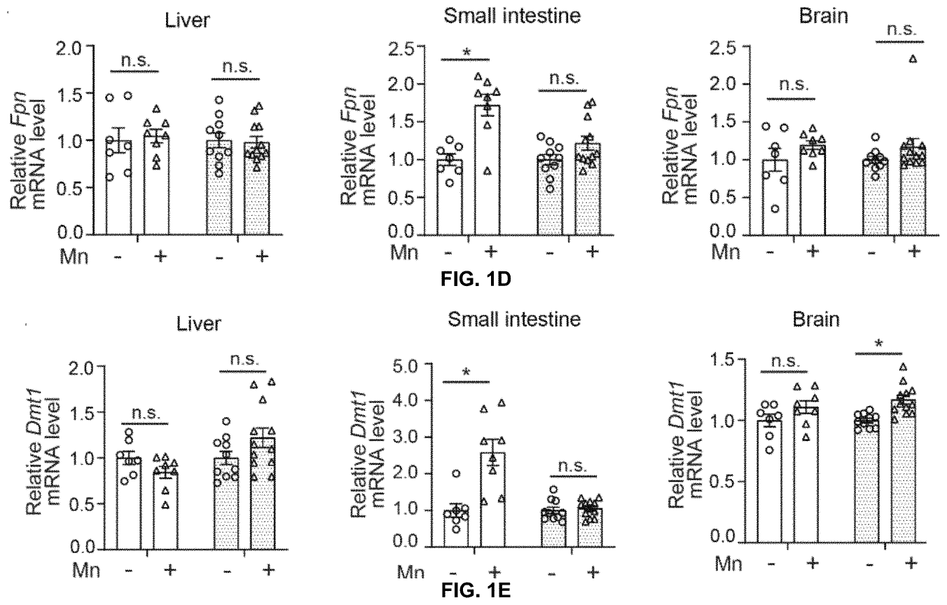

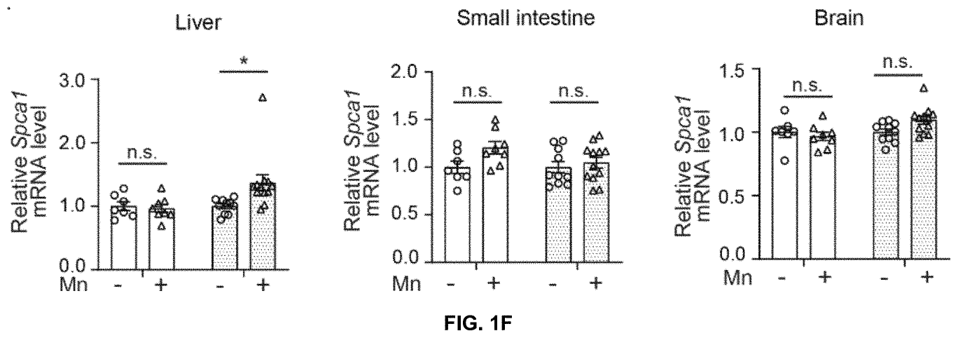

Manganese exposure upregulates SLC30A10 in the mouse liver or intestine. To test that manganese homeostasis may be regulated by changes in critical manganese transporters, mice were exposed to a daily drinking water-based manganese exposure regimen that models environmental manganese exposure in humans (11). In consideration of strain-specific effects, this experiment was performed in congenic 129S4/SvJaeJ or C57BL/6J mice, which are widely used in biomedical research. Epidemiological studies indicate that neurotoxicity from environmental manganese exposure primarily affects human children and adolescents (14-24). To recapitulate exposure across these developmentally-sensitive early-life periods, mice were dosed with manganese from birth until 8-weeks of age when they attain adulthood (62) and assayed for changes in transporter expression using qRT-PCR. Manganese treatment increased levels of SLC30A10 in the liver of 129S4/SvJaeJ, but not C57BL/6J, mice (FIG. 1A). In contrast, there was a manganese-induced increase in SLC30A10 levels in the intestines of C57BL/6J, but not 129S4/SvJaeJ, mice (FIG. 1A). The manganese regimen did not impact expression of SLC30A10 in the brain of either strain (FIG. 1A). Hepatic and brain SLC39A14 and SLC39A8 levels were elevated in the 129S4/SvJaeJ strain, while intestinal SLC39A14 levels were enhanced in C57BL/6J mice (FIGS. 1B and C). Intestinal SLC39A8 levels did not change in either strain (FIG. 1C). Modest strain- and organ-dependent changes in two other known manganese transporters—SPCA1, which transports manganese and Ca from the cytosol into the Golgi (63), and the divalent metal importer DMT1 (13), as well as in the Fe efflux transporter Fpn (FIG. 1D-F), was observed. Thus these animal studies show: (i) the manganese-induced upregulation of SLC30A10 and SLC39A14 in the digestive system may be homeostatically important as it was detected in two strains of mice, albeit in different organs; and (ii) strain-specific effects may make it challenging to interpret rodent studies of manganese homeostasis; instead, a comprehensive mechanistic understanding of the homeostatic control of manganese using disease-relevant culture models was necessary before well-controlled animal studies could be designed.

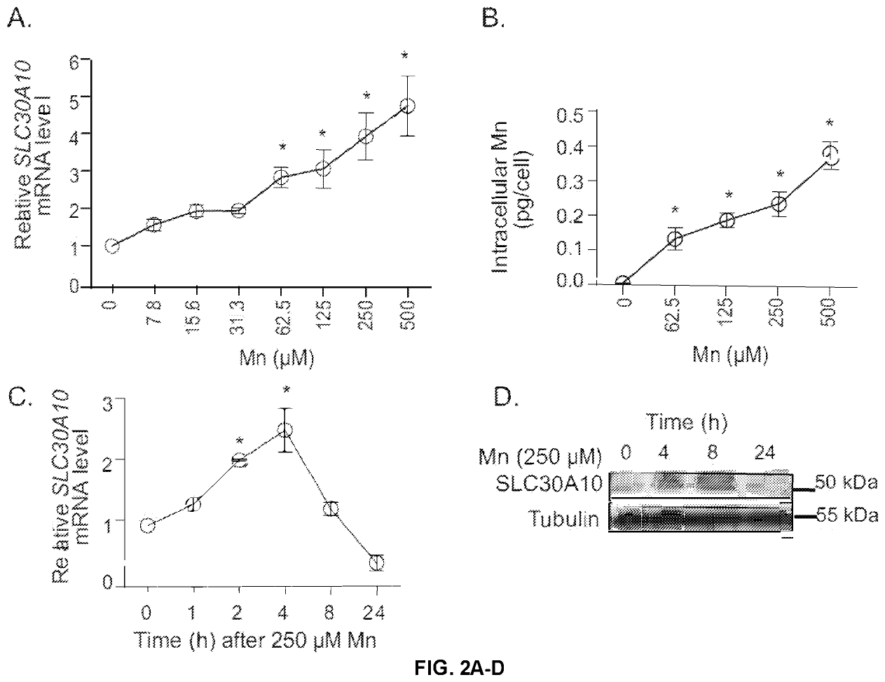

Manganese transcriptionally upregulates SLC30A10. Based on the above, cell-based systems were utilized for subsequent studies. Human-derived HepG2 hepatic cells were used for detailed assays because: (i) the liver is the primary organ that excretes manganese; (ii) a robust manganese-induced increase in hepatic SLC30A10 and SLC39A14 was detected in 129S4/SvJaeJ mice (FIGS. 1 A & B); and (iii) HepG2 cells express SLC30A10 and SLC39A14, and are amenable to genetic manipulation. Subsequently, primary human hepatocytes were used to validate key findings. In HepG2 cells, manganese-treatment significantly elevated SLC30A10, but not SLC39A14, levels (Table 1). Dose response experiments revealed that SLC30A10 levels were elevated in cultures exposed to levels of manganese as low as 62.5 μM (FIG. 2A), which produced intracellular manganese levels equivalent to ˜0.1 pg/cell (compared to ˜1 fg/cell in vehicle-treated control cells) (FIG. 2B). Time-course assays showed that manganese rapidly upregulated SLC30A10, with statistically significant increases evident within 2 h, and levels returned to baseline by 24 h (FIG. 2C). Immunoblot analyses confirmed that manganese increased SLC30A10 protein levels (FIG. 2D). Overall, these results: (i) identify the upregulation of SLC30A10 to be a primary response to elevated manganese exposure in hepatic systems in vitro; (ii) indicate that SLC30A10 expression is highly sensitive to cellular manganese levels with increases in manganese causing rapid elevations in SLC30A10; and (iii) are consistent with the effects of manganese on SLC30A10 expression observed in preceding mouse studies.

TABLE 1
manganese upregulates SLC30A10 and VEGF expression in HepG2 cells.

| | Relative expression | t-test in comparison |
|---|---|---|
| Transcript | after manganese | with vehicle |
| SLC30A10 | 2.40 ± 0.28 | p < 0.05 |
| SLC39AI4 | 0.71 ± 0.02 | p < 0.05 |
| SLC39A8 | 0.87 ± 0.09 | n.s. |
| SPCA1 | 1.00 + 0.05 | n.s. |
| FPN | 0.77 ± 0.21 | n.s. |
| DMT1 | 0.92 ± 0.03 | p < 0.05 |
| VEGF | 2.41 ± 0.29 | p < 0.05 |

Cells were treated with 0 or 250 μM manganese for 4 h, and gene expression was analyzed by qRT-PCR. For each transcript, expression without manganese treatment was normalized to 1. N=4. Data are mean±SE.

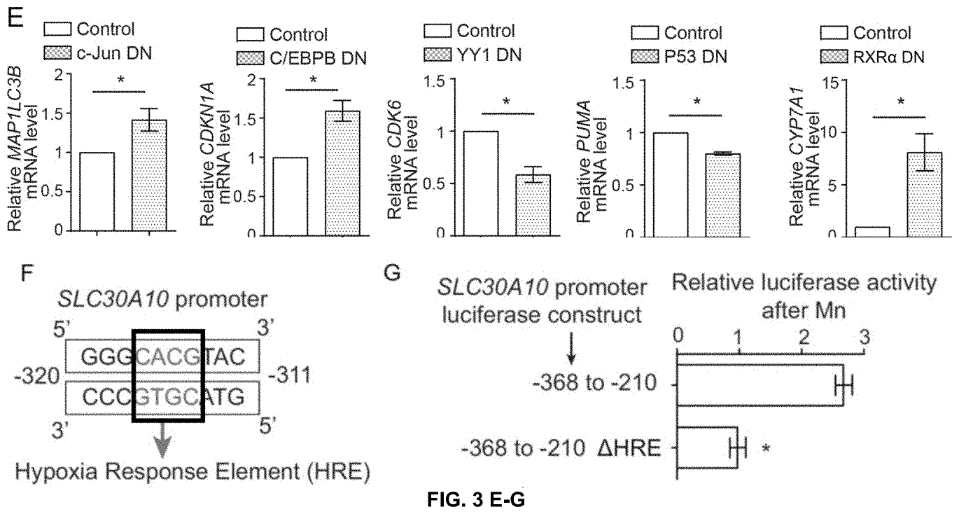

SLC30A10 is transcriptionally upregulated by manganese, and a hypoxia response element in the SLC30A10 promoter is required. The mechanisms of the manganese-induced upregulation of SLC30A10 were studied. Pre-treatment with the transcription inhibitor actinomycin D (64) blocked the increase in SLC30A10 mRNA after manganese exposure in HepG2 cells (FIG. 3A), indicating that transcriptional activity was required for the SLC30A10 response, and that SLC30A10 gene expression may be transcriptionally upregulated by elevated manganese. To test this, a promoter-reporter approach was used. The promoter of SLC30A10 (nucleotides −679 to +211 relative to the start of transcription of SLC30A10 gene) was fused upstream of the luciferase gene (this construct is referred as SLC30A10^{-679 to +211}luc) (FIG. 3B) and lentivirus was used to generate HepG2 cells that stably expressed this construct. Treatment with manganese increased luciferase activity in cells expressing SLC30A10^{-679 to +211}luc, while as expected, there was no change in cells expressing a luciferase-only control construct (FIGS. 3B & C). Thus, the promoter of SLC30A10 contains a sequence element that responds to elevated manganese by increasing downstream gene transcription. To identify this sequence element, aspects of the promoter in SLC30A10^{-679 to +211} luc (FIG. 3B) was deleted, HepG2 cells were stably infected with the generated constructs, and the luciferase assay was repeated. The manganese-induced increase in luciferase activity was evident in cells expressing SLC30A10^{-679 to -12} luc or SLC30A10^{-579 to -210} luc, but not SLC30A10^{-679 to -449}luc (FIGS. 3B & C), suggesting that residues −448 to −210 contained the manganese responsive element. Indeed, SLC30A10^{Δ-448 to -210}luc failed to respond to manganese, while SLC30A10^{-448 to -210}luc exhibited a robust response (FIGS. 3B & C). Thus, residues −448 to −210 of the SLC30A10 promoter are required and sufficient to mediate the manganese-induced increase in transcription. Through a subsequent set of deletion analyses, the required and sufficient sequence was refined to residues −368 to −210 in the SLC30A10 promoter (FIGS. 3B&C). Bioinformatic analyses using the PROMO-ALGGEN algorithm (65, 66) revealed that the −368 to −210 region contained putative binding sites for C/EBPB, YY1, RXRα, HIF, P53, and c-Jun transcription factors. Expression of dominant negative versions of C/EBPB, YY1, RXRα, P53, or c-Jun did not affect the manganese-induced upregulation of SLC30A10 mRNA (FIG. 3D). As a positive control, it was verified that the dominant negative mutants produced expected changes in the expression of their known target genes (67-71)—increase in MAP1LC3B, CDKN1A, or CYP7A1 mRNA with dominant negative C-Jun, C/EBPB or RXRα respectively and decrease in CDK6 or PUMA mRNA with dominant negative YY1 or P53 respectively (FIG. 3E). Thus, the manganese-induced upregulation of SLC30A10 is independent of C/EBPB, YY1, RXRα, P53, and c-Jun. A dominant negative approach to inhibit HIF could not be used (see knockdown studies below). Instead, the only hypoxia response element within the −368 to −210 region of the SLC30A10 promoter (5′RCGTG (72)), which is the binding site for HIF transcription factors was deleted (FIG. 3F), and repeated the luciferase assay. Importantly, deletion of this hypoxia response element abolished the manganese-induced increase in luciferase activity (FIG. 3G). In totality, manganese transcriptionally upregulates SLC30A10, and a hypoxia response element in the promoter of SLC30A10 is required.

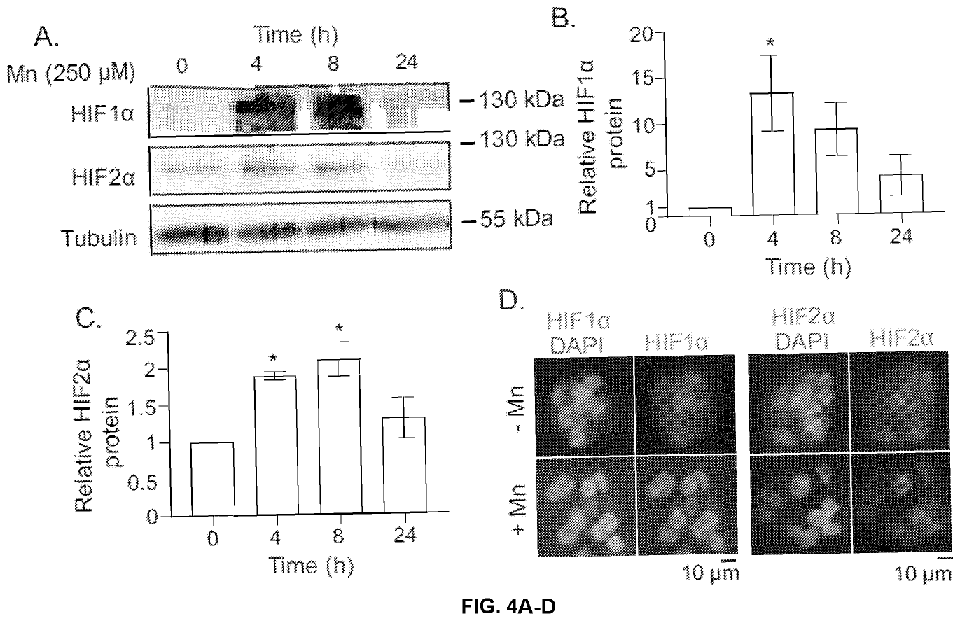

Transcriptional activity of HIF1 or HIF2 is required and sufficient to increase SLC30A10 expression after manganese exposure. Results presented above suggest that HIF transcription factors likely mediate the manganese-induced upregulation of SLC30A10. HIFs are heterodimeric transcription factors formed by the association of an a subunit, the most well-studied of which are HIF1α or HIF2α, with a common β subunit (HIF1β/ARNT) (72). The transcriptionally active moieties are named after the a subunits. Both HIF1 and HIF2 bind the canonical hypoxia response element (73). Under normoxic conditions, HIFα sub-units are rapidly hydroxylated by prolyl hydroxylases, subsequently bound by the von Hippel-Lindau (VHL) protein complex, ubiquitinated, and targeted for proteasomal degradation (72, 74). Hypoxia inhibits prolyl hydroxylases, leading to an increase in HIF1α or HIF2α protein levels and allowing for the formation of transcriptionally active heterodimeric complexes with HIF1β/ARNT (72, 74). Notably, elevated levels of divalent metals also inhibit prolyl hydroxylases, which are Fe containing enzymes, and increase HIF1α protein levels as well as HIF1-dependent transcription (75-81). Thus, whether elevated manganese may induce SLC30A10 expression by activating HIFs were tested. But, before directly testing this, it was essential to confirm that HIFs were indeed activated in the studied experimental system. It was observed that treatment with manganese increased protein levels of HIF1α and HIF2α in HepG2 cells (FIG. 4A-C). The increase was evident at 4 hours and returned to baseline by 24 h (FIG. 4A-C), similar to the time-course of the SLC30A10 response (FIG. 2). Further, HIF1α and HIF2α were detected in the nucleus of manganese-treated cells (FIG. 4D), which is necessary for transcriptional activity. Finally, manganese also enhanced expression of VEGF (Table 1), a canonical HIF1 target gene (72). These findings indicated that manganese treatment activated HIF transcription factors, and combined with the luciferase-reporter assays, provided strong justification to test for the requirement of HIF1 or HIF2 for the manganese-induced SLC30A10 response.

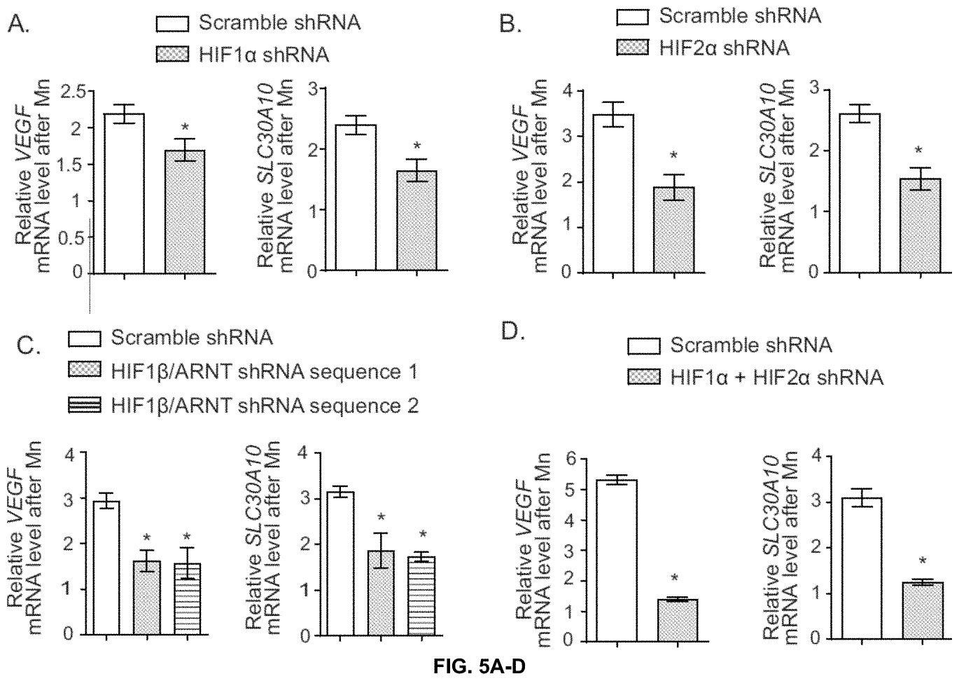

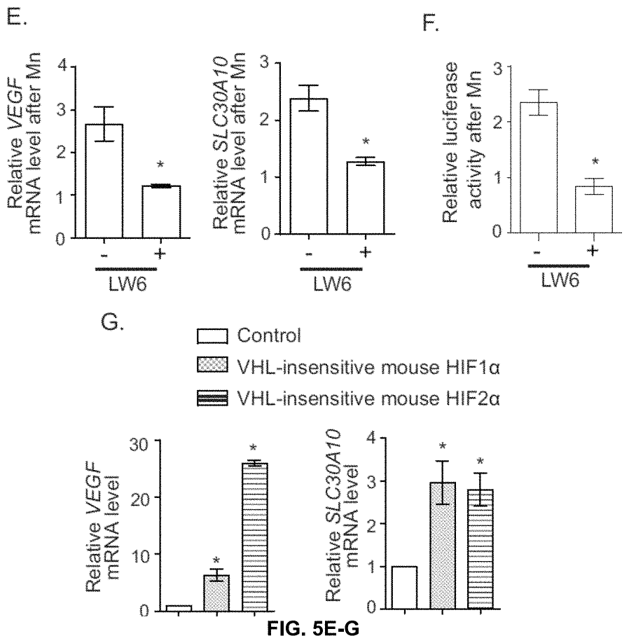

To test for the role of HIF1 or HIF2, a lentivirus-based shRNA system was used to stably knockdown HIF1α or HIF2α in cells. Both shRNAs significantly depleted the targeted gene product (Table 2); a compensatory increase in HIF1α mRNA was detected with HIF2α knockdown (Table 2). However, despite efficient knockdown, the manganese-induced elevation of VEGF or SLC30A10 was only modestly attenuated (FIGS. 5A&B). The lack of a strong inhibitory effect of knockdown of either gene product could be due to redundancy. To test this, the common HIF1β/ARNT subunit was depleted using two separate shRNAs. Each shRNA had a knockdown efficiency of ˜50% (Table 2), and each shRNA partially inhibited the manganese-induced elevation of VEGF and SLC30A10 by ˜50-60% relative to scramble (FIG. 5C). The failure of HIF1β/ARNT knockdown to also completely abolish the SLC30A10 response could be a consequence of incomplete knockdown or indicate that another transcription factor was involved. Therefore, in subsequent experiments, HIF1α and HIF2α double knockdown cells were generated by performing sequential lentiviral infections. Knockdown efficiency of HIF1α was ˜80% and of HIF2α was ˜65% (Table 2). Notably, the ability of manganese to enhance SLC30A10 expression was very strongly repressed in the double knockdown cells, and relative to scramble shRNA, the manganese-induced increase of SLC30A10 was inhibited by ˜90% in the double knockdown cells (FIG. 5D). A similar strong inhibition of the manganese-induced elevation of VEGF was also observed (FIG. 5D). For further validation, the small molecule LW6, which degrades HIFα subunits by inducing expression of VHL (82), was used. Importantly, LW6 treatment robustly blocked the manganese-induced upregulation of SLC30A10 and VEGF (FIG. 5E), and further, LW6 also abolished the manganese-induced increase in the activity of the SLC30A10 promoter luciferase reporter (FIG. 5F). Put together, data from the knockdown and LW6 assays indicate that activity of HIF1 or HIF2 is obligatorily required for the manganese-induced upregulation of SLC30A10. Finally, expression of VHL-insensitive versions of mouse HIF1α or HIF2α that could not get prolyl hydroxylated and were therefore expressed at high levels under normoxic conditions increased SLC30A10 levels independent of manganese exposure (FIG. 5G), implying that activity of either HIF1 or HIF2 is sufficient to upregulate SLC30A10. Overall, these results indicate that the manganese-induced transcriptional upregulation of SLC30A10 is mediated by HIF1 and HIF2 in a redundant manner.

TABLE 2
Validation of shRNAs targeting HIF1α, HIF2α or HIF1β/ARNT.

| | | Relative expression | t-test or ANOVA in |
|---|---|---|---|
| | | in comparison with | comparison with |
| shRNA | Transcript | scramble shRNA | scramble shRNA |
| HIF1α | HIF1α | 0.16 ± 0.02 | p < 0.05 |
| HIF1α | HIF2α | 1.13 ± 0.13 | n.s. |
| HIF2α | HIF1α | 1.42 ± 0.11 | p < 0.05 |
| HIF2α | HIF2α | 0.29 ± 0.07 | p < 0.05 |
| HIF1β/ARNT | HlF1β/ARNT | 0.59 ± 0.03 | p < 0.05 |
| sequence 1 | | | |
| HIF1βARNT | HIF1β/ARNT | 0.47 ± 0.02 | p < 0.05 |
| sequence 2 | | | |
| HIF1α + HIF2α | HIF1α | 0.20 ± 0.01 | p < 0.05 |
| HIF1α + HIF2α | HIF2α | 0.35 ± 0.02 | p < 0.05 |

qRT-PCR analyses in HepG2 cells stably infected with scramble shRNAs or shRNAs targeting HIF1α, HIF2α, HIF1β/ARNT or HIF1α+HIF2α. Expression in respective scramble shRNA control cells was normalized to 1. Scramble shRNA sequence 1 was used for the HIF1α shRNA assay (single knockdown). Scramble shRNA sequence 2 was used for the other experiments, including HIF1α+HIF2α double knockdown. N=3-4. Except HIF1β/ARNT shRNAs, p<0.05 by t-test in comparison with respective scramble shRNA. For HIF1β/ARNT shRNAs, p<0.05 by one-way ANOVA and Dunnett's post hoc test in comparison with scramble shRNA. N.S.—not significant.

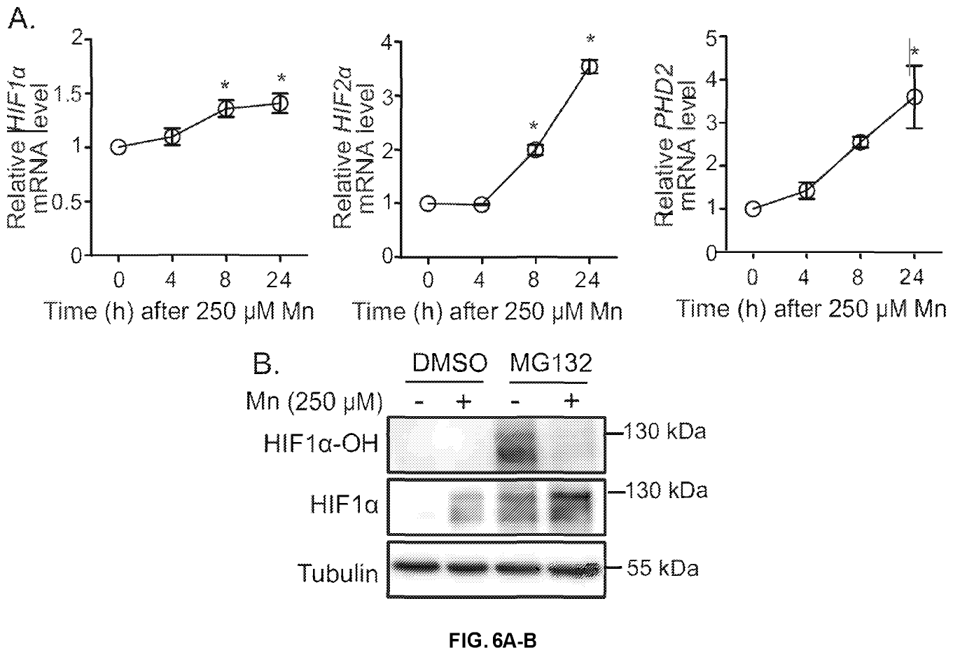

Manganese inhibits the prolyl hydroxylation of HIFα subunit. To elucidate the mechanism by which manganese increased HIFα protein levels, changes in HIF1α prolyl hydroxylation in HepG2 cells were assayed because: (i) metals are known inhibitors of the prolyl hydroxylation of HIFα subunits (77-80); (ii) although manganese increased HIF1α or HIF2α protein levels by 4 h (FIG. 4A), levels of HIF1α or HIF2α mRNA were not elevated at this time-point (FIG. 6A), suggesting that the increase in HIF1α and HIF2α protein was post-translational; and (iii) a specific and sensitive antibody against human hydroxy-prolyl HIF1α was available. Since prolyl hydroxylated HIF1α is rapidly degraded in the proteasome (72, 74), the assay was performed with or without treatment with the proteasome inhibitor MG132. In the absence of MG132, prolyl hydroxylated HIF1α was not detected irrespective of manganese treatment (FIG. 6B), but similar to results in FIG. 4A, total HIF1α levels were elevated after manganese treatment (FIG. 6B). Proteasome inhibition led to the accumulation of prolyl hydroxylated HIF1α in cells that had not been exposed to manganese (FIG. 6B). Importantly, however, signals for prolyl hydroxylated HIF1α were substantially lower in the manganese-treated condition, and total HIF1α levels of manganese-treated cells were greater than in those not treated with manganese (FIG. 6B). Finally, levels of prolyl hydroxylase domain 2 (PHD2), the primary enzyme that hydroxylates HIFα subunits (74), were elevated in manganese-treated cells (FIG. 6A), ruling out the possibility that the observed reduction in prolyl hydroxylation of HIF1α was a consequence of a decrease in prolyl hydroxylase gene expression. Thus, manganese inhibits prolyl hydroxylation of HIFα subunits, which increases HIFα protein levels and induces HIF-dependent transcription.

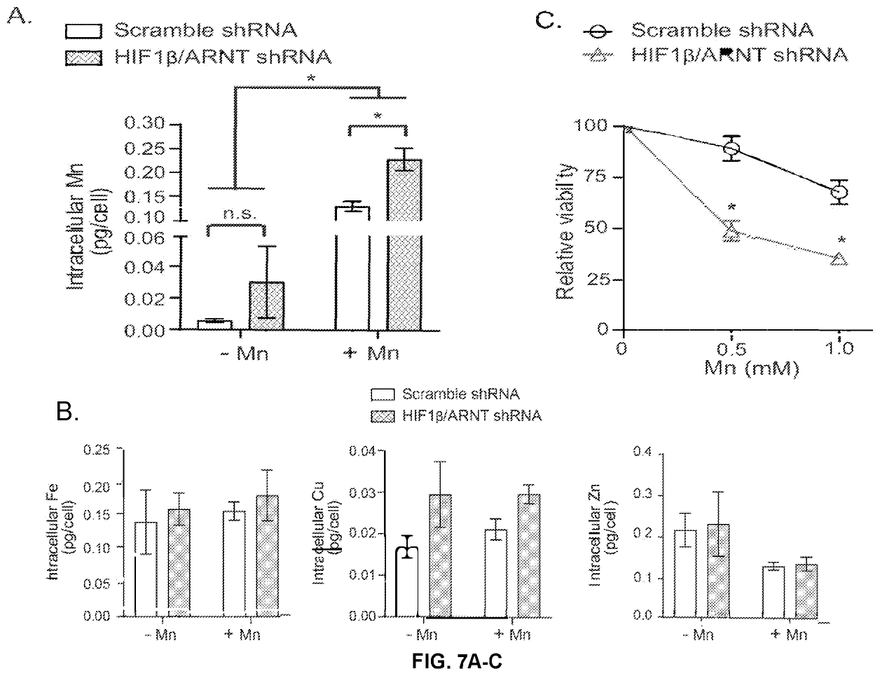

The manganese-induced upregulation of SLC30A10 is a critical, homeostatic protective response. To determine the physiological relevance of the manganese-induced increase in SLC30A10, intracellular metal levels and viability of HIF1β/ARNT knockdown HepG2 cells, in which the SLC30A10 response is inhibited, was compared with controls. As both HIF1β/ARNT shRNAs were equally effective in inhibiting the SLC30A10 response (FIG. 5C), the metal measurement and viability assays were performed using only one shRNA. Under basal conditions, manganese levels of the knockdown cells were comparable to scramble shRNA-infected control (FIG. 7A). However, after manganese treatment, manganese levels in HIF1β/ARNT knockdown cells were higher than controls (FIG. 7A). Levels of other metals (Fe, Cu, and Zn) were unaltered (FIG. 7B). Further, HIF1β/ARNT knockdown cells were more sensitive to manganese-induced death (FIG. 7C). These results suggest that the upregulation of SLC30A10 is a critical homeostatic response to elevated manganese exposure that reduces intracellular manganese levels and protects against manganese toxicity.

### Example 2

### Prolyl Hydroxylase Inhibitors Protect Against Manganese Toxicity

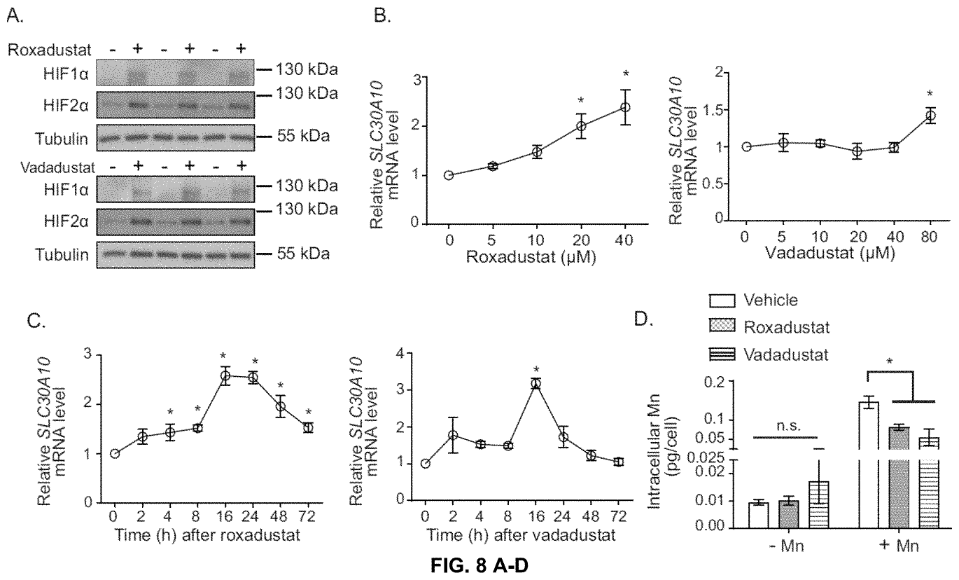

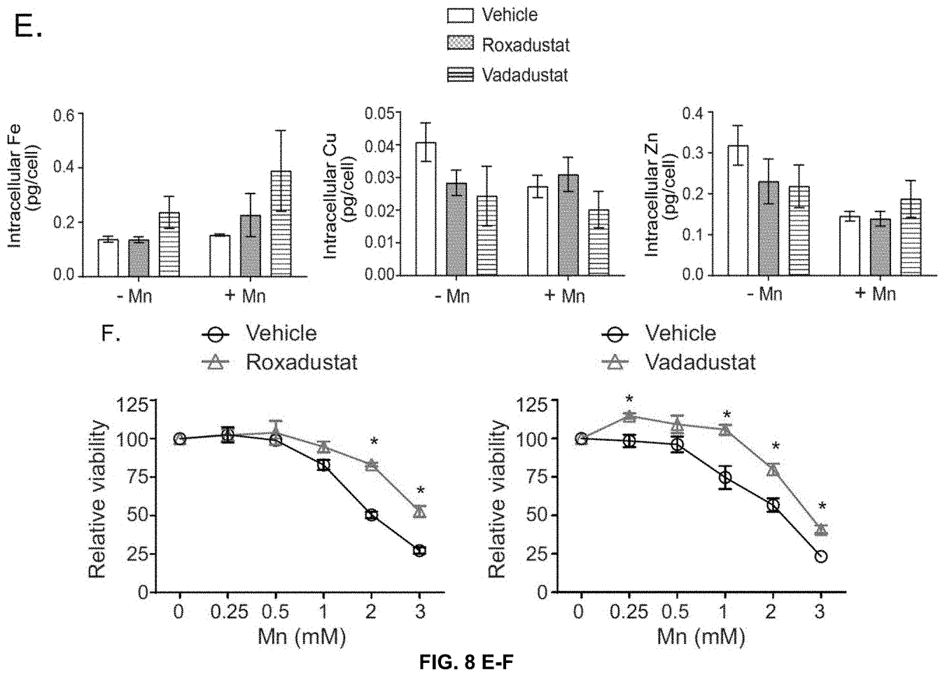

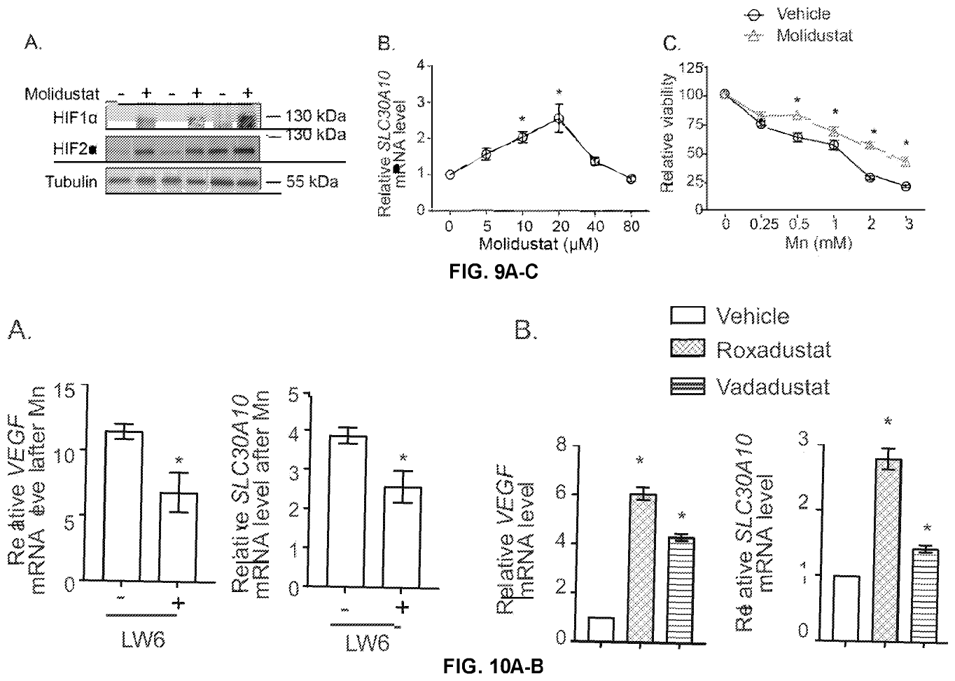

Prolyl hydroxylase inhibitors protect cells against manganese toxicity. Roxadustat, vadadustat and molidustat are small molecule inhibitors of prolyl hydroxylases that have completed, or are in, advanced clinical trials for the treatment of renal anemia (83-86). By inhibiting prolyl hydroxylase enzymes, these compounds increase HIFα protein and activate HIF-dependent transcription (83-86). Inventors have discovered that SLC30A10 is upregulated by HIF1/HIF2, showing that prolyl hydroxylase inhibitors may induce SLC30A10 expression, reduce manganese levels, and protect against manganese toxicity. In HepG2 cells, roxadustat or vadadustat increased levels of HIF1α and HIF2α protein (FIG. 8A), and enhanced expression of SLC30A10 in a concentration- and time-dependent manner (FIGS. 8B&C). Further, while roxadustat or vadadustat did not impact intracellular manganese in cells not exposed to manganese, under conditions of elevated manganese exposure, intracellular manganese levels of cells treated with roxadustat or vadadustat were significantly lower than controls (FIG. 8D). Levels of other metals (Fe, Cu and Zn) were not altered by drug treatment (FIG. 8E), indicating that the effect was specific to manganese. Roxadustat or vadadustat also protected cells against manganese-induced cell death (FIG. 8F). Similar results were obtained with molidustat (FIG. 8G-I). Thus, prolyl hydroxylase inhibitors increase SLC30A10 expression, reduce intracellular manganese levels, and protect cells against manganese toxicity.

Validation of results in primary human hepatocytes. To validate the physiological relevance of results obtained in HepG2 cells, confirmatory assays in primary human hepatocytes were performed. Importantly, in the primary system, treatment with manganese increased VEGF or SLC30A10 expression, and this increase was inhibited by the HIF-inhibitor LW6 (FIG. 10A). Additionally, treatment with roxadustat or vadadustat also increased VEGF or SLC30A10 expression (FIG. 10B). These results validate the primary conclusions of findings from HepG2 cells and indicate that manganese increases SLC30A10 expression in a HIF-dependent manner in primary hepatocytes.

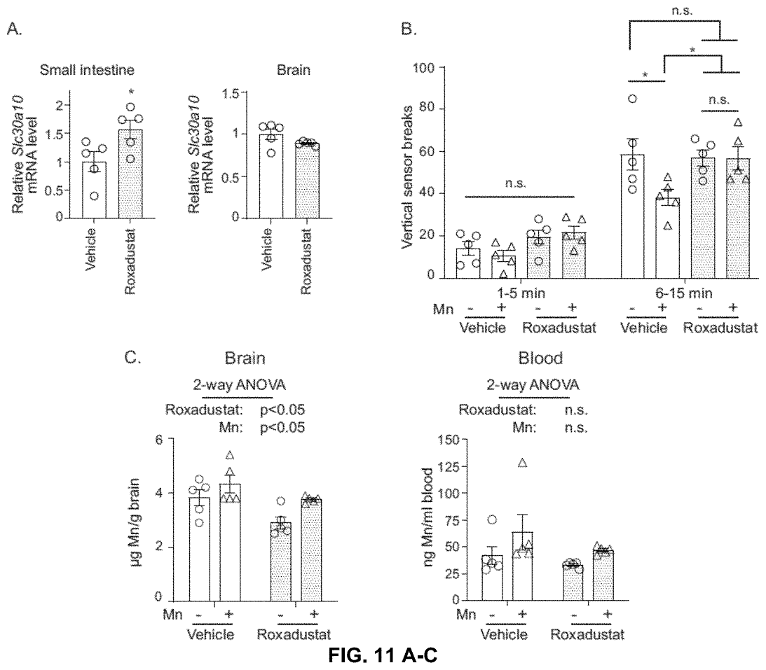

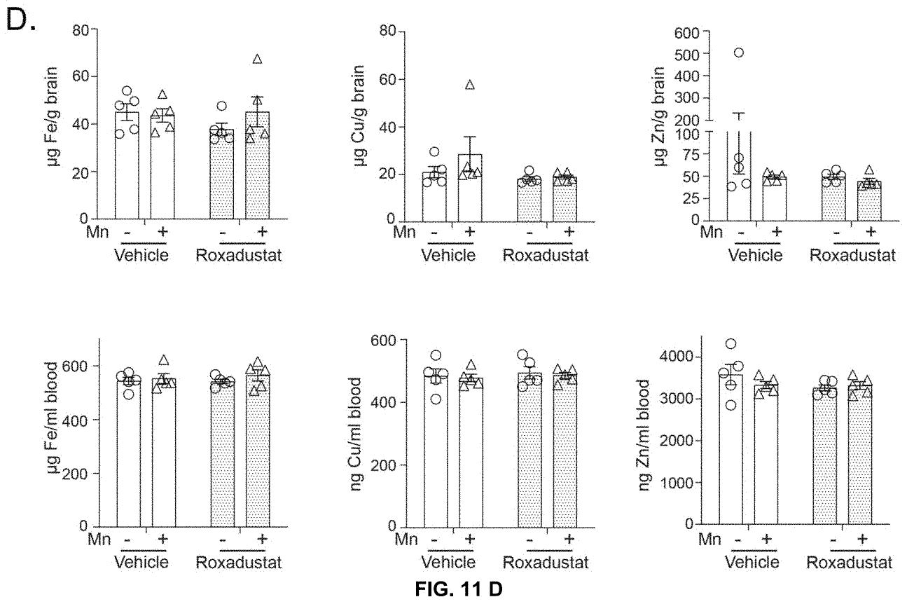

Prolyl hydroxylase inhibitors protects mice against manganese neurotoxicity. Based on the compelling cell culture data, analyses were expanded to mice and performed a proof-of-principle protection experiment using roxadustat. C57BL/6J mice were used for this assay because: (i) neurobehavioral outcomes are well characterized in this strain; (ii) inventors observed that roxadustat treatment increased intestinal SLC30A10 levels in these animals (FIG. 11A); and (iii) similar to an increase in hepatic SLC30A10, increased intestinal SLC30A10 is expected to enhance manganese excretion. Neurological function was assayed for using the open-field test. The first 5 min interval of the test provides information about behavioral reactivity and exploratory behavior of rodents in a novel environment, and increases in activity in this interval may be indicative of anxiety-like disorders. The succeeding 10 min interval is a measure of the generalized locomotor activity of the animals, and decreases in this interval are indicative of neuromotor deficits. There were no differences between groups in the first 5-min interval (FIG. 11B). In the 6-15 min interval, consistent with a neuromotor dysfunction, animals exposed to manganese-only exhibited reduced vertical (i.e. rearing) movement than vehicle-treated controls (FIG. 11B). Importantly, this hypo-locomotor phenotype was not detected in mice treated with manganese and roxadustat (FIG. 11B), and additionally, roxadustat treatment by itself did not impact activity (FIG. 11B). Tissue metal measurements performed after completion of the neurobehavioral test revealed that, as expected, there was a main effect of manganese treatment in increasing brain manganese levels (FIG. 11C), and a trend towards an increase in blood manganese was also evident (FIG. 11C). Notably, there was a main effect of roxadustat in reducing brain manganese levels (FIG. 11C), providing an explanation for the neuroprotective effect of roxadustat in the open field test. Changes in manganese were specific because levels of other metals (Fe, Cu, and Zn) were not impacted by manganese exposure or roxadustat treatment (FIG. 11D). Roxadustat did not impact brain SLC30A10 levels (FIG. 11A), suggesting that the protective effect of roxadustat against manganese neurotoxicity was unlikely to be a consequence of a direct reduction of brain manganese levels by elevations in SLC30A10, and instead, more likely to be reflective of an increase in manganese excretion. Overall, roxadustat reduces brain manganese levels and protects against manganese neurotoxicity, suggesting that it may be useful for the treatment of neurological disease induced by manganese in humans.

### Methods Used for the Experiments in the Examples

Animal experiments. All experiments with mice were approved by the Institutional Animal Care and Use Committee of the University of Texas at Austin.

A well-characterized drinking water-based manganese exposure regimen was used to model human environmental manganese exposure by providing mice with ˜50 mg manganeseCl₂.4H₂O/kg body weight daily (˜15 mg absolute manganese/kg per day) from birth until 8 weeks of age. This, and related similar, dosing regimens: 1) approximate the increase in manganese exposure in humans consuming well water contaminated with 1.5 mg manganese/L, which is the median well water concentration associated with neurological deficits in children; 2) increase brain manganese levels by ˜2-3 fold, similar to human patients; 3) produce measurable neurobehavioral deficits without overt toxicity; and 4) model exposure to manganese in developmentally-sensitive early-life and adolescent periods (11). Pre-weaning manganese was delivered by pipette directly into the mouth, and post-weaning manganese was delivered in drinking water using procedures described in detail in ref. (11). In brief, pre-weaning dosing was adjusted to each animal's body weight. For this, a stock solution of 69.2 mg absolute manganese/ml prepared in milliQ water was diluted in milliQ water containing 2.5% (wt/vol) of the natural sweetener Stevia (to facilitate intake by pups), and the required manganese amount (0.2 μl manganese stock/g body weight) was delivered in a dose of ˜5-10 μl/animal. For post-weaning exposure, a 27.7 mg absolute manganese/ml stock solution was prepared in water and diluted to 0.069 mg absolute manganese/ml in drinking water, which provided mice with ˜15 mg absolute manganese/kg per day based on a daily water intake of ˜2-5 ml/mouse depending on age.

To assay for the protective effect of roxadustat, animals were exposed to roxadustat or vehicle with or without manganese in drinking water from ˜4 weeks of age. Treatment lasted for 4 weeks from initiation. Roxadustat (Selleckchem) was prepared as a 50 mg/ml stock solution in DMSO, then diluted to 1 mg/ml in sterile phosphate buffered saline (PBS), and delivered by daily i.p. injection at 10 mg/kg. Vehicle was 2% DMSO in PBS. For this experiment, drinking water manganese levels were adjusted to 0.138 mg absolute manganese/ml so that animals received ˜30 mg absolute manganese/kg daily. A higher level of manganese was used because exposure was initiated after weaning, which was necessary because daily i.p. injections for roxadustat delivery were not possible in the pre-weaning period.

Breeders for C57BL/6J and 12654/SvJaeJ mice were obtained from The Jackson Labs. Mice were crossed and litters housed in the conventional facility of the University of Texas at Austin in a room maintained at 21° C. with a 12-h light-dark cycle (lights on between 7 p.m. and 7 a.m.). Animals were weaned at PND 21. After weaning, 3-4 littermates of the same sex were kept per cage. Animals had free access to food and water.

Animals were euthanized using carbon dioxide and tissue dissected for qRT-PCR or ICPMS analyses as described by us previously (50).

Cell culture. HepG2 cells were grown in Eagle's modified essential medium (MEM; Corning) with 10% fetal bovine serum (Atlanta Biologicals), 100 IU/ml penicillin (Corning), and 100 μg/ml streptomycin (Corning). 293T cells were maintained in Dulbecco's modified essential medium/F12 (Thermo) supplemented with 10% fetal bovine serum, penicillin (100 IU/ml), and streptomycin (100 μg/ml). Human primary hepatocytes were purchased from XenoTech (catalog #HPCH10+) and cultured according to protocols provided by the supplier.

manganese and drug treatments in cell culture. Cells were treated with indicated levels of manganese as manganeseCl₂.4H₂O (Fisher). Vehicle for manganese treatment was water. Prolyl hydroxylase inhibitors roxadustat (Caymen), vadadustat, and molidustat (both APE-Bio); the HIFα inhibitor LW6 (Selleckchem); the proteasomal inhibitor MG132 (Sigma); and the transcription inhibitor actinomycin D (Sigma) were dissolved in DMSO. LW6, MG132, and actinomycin D were used at an effective concentration of 10 μM, 10 μM, and 5 μM, respectively (64, 82) while prolyl hydroxylase inhibitors were used at concentrations indicated in the figure legends. Vehicle treatment for all drugs was DMSO.

Plasmids and shRNAs. Lentivirus infections were used to stably express luciferase or dominant negative constructs, and shRNAs targeting HIF1α, HIF2α, or HIF1β/ARNT.

A luciferase reporter plasmid containing the proximal promoter of human SLC30A10 has been previously described and was a gift of Dr. Ruth Valentine (100). The promoter fragment corresponding to bases −679 to +211 relative to the start of transcription was sub-cloned into the Xho1 and SpeI sites of pRRLSIN.cPPTLuciferase.WPRE vector (Addgene, #69251), which is a luciferase vector for lentivirus infections. Deletion mutants were generated using the loop-out modification of the QuikChange protocol.

Plasmids encoding dominant negative versions of C/EBPB, which contains an N-terminal acidic extension fused to the HLH-ZIP region, c-Jun encoding amino acid residues 123-331, and P53 encoding a truncated protein of amino acids 300-393 were obtained from Addgene (#33363, 40350, and 25989 respectively). Plasmids encoding full-length RXRα or YY1 were obtained from DNASU (#HsCD00079702 or #HsCD00005306, respectively), and dominant negative versions generated by deleting the transactivation domain (amino acids 444-462) of RXRα (101) or introducing the S339/S342 mutation in YY1 (102). Plasmids encoding VHL insensitive mouse HIF1α and HIF2α were a gift of Dr. Sadeesh Ramakrishnan (University of Pittsburgh). These open reading frames were sub-cloned into the Nhe1/EcoR1 sites of the lentivirus transfer plasmid LAMP1-mRFP-FLAG (Addgene #34611), which has been described previously (49).

ShRNA sequences targeting HIF1α, HIF2α, or HIF1β/ARNT (Table 3) were obtained from the MISSION® TRC shRNA library (Sigma) or were previously described (103). Scramble shRNA sequence 1 was from the MISSION® TRC shRNA library while scramble shRNA sequence 2 (Table 3) has been previously reported (104). These sequences were sub-cloned into the lentivirus transfer plasmid pLKO.1 vector (Addgene #8453).

TABLE 3
Sequences of shRNAs used. For HIF1β/ARNT and
scramble shRNAs, the numbers 1 and
2 refer to sequence 1 and 2.

| shRNA | Sequence | Reference |
|---|---|---|
| HIF1α | TGCTCTTTGTGGTTGGATCTA | (103) |
| HIF2α | CAGTACCCAGACGGATTTCAA | TRCN0000003806 |
| | | (MISSION® shRNA |
| | | library (Sigma)) |
| HIF1β | AGCCTCATCATCGTTCAAGTT | TRCN0000003820 |
| shRNA-1 | | (MISSION® shRNA |
| | | library (Sigma)) |
| HIF1β | GCCTACACTCTCCAACACAAT | TRCN0000003816 |
| shRNA-2 | | (MISSION® shRNA |
| | | library (Sigma)) |
| Scramble | CAACAAGATGAAGAGCACCAA | SHC002 |
| shRNA-1 | | (MISSION® shRNA |
| | | library (Sigma)) |
| Scramble | GTGGACTCTTGAAAGTACTAT | (104) |
| shRNA-2 | | |

Lentiviral infections. Stable cells were generated by lentivirus infection essentially as described by us previously (49, 98). Briefly, lentivirus was produced in HEK293T cells by co-transfecting transfer plasmids described above with plasmids coding for a third generation packing system that is routinely used (pRSV-Rev, pRRE, and pCMV-VSVG) (49, 98). Viral supernatants were collected 40-48 h post-transfection. HepG2 cells for infection were plated in a 60 mm dish 24 h before infection so that cells reached a confluence of 30% on the day of infection. Collected viral supernatants were filtered through a 0.45-μm polyvinylidene difluoride filter, and cells were incubated with a 1:2 dilution of viral supernatant containing 5 μg/mL Polybrene (Santa Cruz). Media was changed the next day to fresh MEM supplemented with 10% fetal bovine serum, penicillin (100 IU/ml), and streptomycin (100 μg/ml). Forty-eight hours after infection, for dominant negative and shRNA-infections, 2 μg/mL puromycin (Sigma-Aldrich) was added to obtain puromycin-resistant cells (the transfer plasmid for luciferase constructs did not confer resistance to puromycin). For HIF1α+HIF2α double knockdown, cells were sequentially infected with lentivirus targeting HIF1α and HIF2α, and the infections were spaced by one week. Control infection cells for the double knockdown were exposed to lentivirus containing scrambled shRNA followed one week later by lentivirus lacking a transfer plasmid.

Quantitative RT-PCR assays. RNA extraction from HepG2 cells and mouse tissue, reverse transcription to cDNA, and qRT-PCR analyses were performed as described by us previously (47, 50). For analyses of mouse brain samples, a part of the midbrain, including the basal ganglia, was used. Transcript levels were quantified using the ΔΔC_{T} method with 18S (in mice) or TBP (in HepG2 cells) as the internal control, also as described by us previously (47, 50). Primers used are listed in Table 4.

TABLE 4
qRT-PCR primers.

| | | Forward primer | Reverse primer |
|---|---|---|---|
| Gene | species | sequence | sequence |
| HIF1α | Human | CCACAGG | TCAAGTC |
| | | ACAGTAC | GTGCTGA |
| | | AGGATG | ATAATAC |
| | | | C |
| HIF2αa | Human | GCGACAA | CAGCATC |
| | | TGACAGC | CCGGGAC |
| | | TGACAA | TTCT |
| HIF1β | Human | CTAGTGG | CAATGTT |
| | | CCATTGG | GTGTCGG |
| | | CAGATT | GAGATG |
| SLC39A8 | Human | ATGCTAC | CAGGAAT |
| | | CCAAATA | CCATATC |
| | | ACCAGC | CCCAAAC |
| DMT1 | Human | TGGCTTA | CACACTG |
| | | TCTGGGC | GCTCTGA |
| | | TTTGTG | TGGCTA |
| SLC39A14 | Human | TGTCTCC | GGAATCA |
| | | AAGTCTG | TGTGGTC |
| | | CAGTGG | CAGGTC |
| SLC30A10 | Human | TTCCCGC | ACTGCTA |
| | | TTATCAA | ATTCCAG |
| | | GGAGACC | GCACAGC |
| FPN | Human | CAGTTAA | AAGCTCA |
| | | CCAACAT | TGGATGT |
| | | CTTAGC | TAGAG |
| SPCA1 | Human | GGATAGA | TGAGGAG |
| | | GTTCCTG | CTGTCAC |
| | | CTGACTT | CTTAGA |
| | | AC | |
| VEGF | Human | GGGTCTC | AGGGCAG |
| | | GATTGGA | AATCATC |
| | | TGGCA | ACGAAGT |
| MAP1LC3B | Human | CCGCCGC | GAGTCAG |
| | | CTTTTTG | GGACCTT |
| | | GGTAG | CAGCAG |
| CDK6 | Human | CGCCTAT | TTGGGGT |
| | | GGGAAGG | GCTCGAA |
| | | TGTTC | GGTCT |
| CDKN1A | Human | ACCTGGA | TTAGGGC |
| | | GACTCTC | TTCCTCT |
| | | AGGGTCG | TGGAGAA |
| | | | GAT |
| PUMA | Human | CTGTGAA | AATGAAT |
| | | TCCTGTG | GCCAGTG |
| | | CTCTGC | GTCACA |
| CYP7A1 | Human | CCATAAG | CATCCAT |
| | | GTGTTGT | CGGGTCA |
| | | GCCACG | ATGCTT |
| TBP | Human | CGAACCA | TTTCTTG |
| | | CGGCACT | CTGCCAG |
| | | GATTTTC | TCTGGAC |
| Slc39a14 | Mouse | AAGTCCC | CTGGGAA |
| | | TGCTCGA | TCCAGCT |
| | | CCAC | GCTG |
| Slc39a8 | Mouse | CTCGCCT | GCTTTGC |
| | | TCAGTGA | GTTGTGC |
| | | GGATGT | TTTCTT |
| Dmt1 | Mouse | TCAGAGC | TGTGAAC |
| | | TCCACCA | GTGAGGA |
| | | TGACTG | TGGGTA |
| Slc30a10 | Mouse | GTAGCAG | GTGATGA |
| | | GTGATTC | CCACAAC |
| | | CCTGAAC | CACGGAC |
| Fpn | Mouse | TTGCAGG | TGGAGTT |
| | | AGTCATT | CTGCACA |
| | | GCTGCTA | CCATTGA |
| | | | T |
| Spca1 | Mouse | GACTCTA | CTTCGTC |
| | | GCCCTTG | AGGGTTC |
| | | GTGTTAT | CAGTTT |
| | | G | |
| 18S | Mouse | CATTAAG | GTCGTGG |
| | | GGCGTGG | GTTCTGC |
| | | GGCGG | ATGATG |

Cell viability assays. These were performed using the 3-(4,5-dimethylthiazol-2-yl)-2,5-diphenyl tetrazolium bromide (MTT) reagent (Sigma) as described by us previously (47, 50).

Inductively coupled plasma mass spectrometry. Metal measurements using ICP-MS were performed exactly as described by us previously (47, 50, 51, 99).

Open-field assays for neurobehavior assessment. Open-field analyses were performed as described in a recent publication (51).

Immunofluorescence microscopy. This was also performed as described d previously (47, 50, 51). Briefly, cells were grown on glass coverslips, fixed with 3% paraformaldehyde, and processed for staining as previously reported (47, 50, 51). Nucleus was stained using 4′,6-diamidino-2-phenylindole (DAPI). Imaging was using a Nikon swept field confocal equipped with a four-line high-power laser launch and 60×, 1.4 numerical aperture oil-immersion objective (Nikon). Image capture was with an iXon3 X3 DU897 EM-CCD camera (Andor Technology).

Immunoblots. HepG2 cells were cultured in 35 mm dishes, treated with or without manganese, MG132 and/or prolyl hydroxylase inhibitors, and scrapped using 200 μl of RIPA buffer (50 mM Tris-HCl, pH7.4; 150 mM NaCl; 1Mm EDTA; 1% Triton; 0.25% sodium deoxycholate; protease inhibitors cocktail (Thermo A32953)). Whole-cell lysates were prepared by adding loading buffer (50 mM Tris-HCl, pH 7.4; 2% sodium dodecyl sulfate; 10% glycerol, 0.1 M dithiothreitol; 0.01% (weight/volume) bromophenol blue) and boiling the samples for 5 min. Equal volumes of samples were loaded per lane. Further processing was as described by us previously (47). Images were quantified using ImageQuant TL software (GE).

Luciferase assays. HepG2 cells stably expressing luciferase constructs were treated with or without 250 μM manganese for 16 h. Luciferase assays were performed using the Luciferase Assay System kit (Promega) according to the manufacturer's instruction and normalized to protein content (Bio-Rad protein assay dye, #5000006).

Transcription factor binding prediction. Transcription factors predicted to bind the SLC30A10 promoter were identified using the PROMO-ALGGEN algorithm (65, 66).

Antibodies and chemicals. The inventors used the following commercial primary antibodies in this study: rabbit polyclonal anti-HIF1α (Abcam, Ab2185), rabbit polyclonal anti-HIF2α (Novus, NB100-122), rabbit monoclonal anti-HIF1α proline 564 (Cell signaling, 3434S), and mouse monoclonal anti-tubulin (Sigma, T5168). We have previously described the custom rabbit polyclonal anti-SLC30A10 antibody raised against the C-terminus of human SLC30A10 that detects SLC30A10 in human cell lines (but not in mouse tissue) (50, 51). Sources of chemicals not provided elsewhere were Thermo Fisher Scientific or Sigma-Aldrich.

Statistical analyses. Cell culture experiments were replicated three or more times independently. Animal numbers are provided in the figure legends. Comparisons between multiple groups were performed using one- or two-way ANOVA and appropriate post hoc tests. Comparisons between two groups were performed using Student's t-test. The Prism 8 software (GraphPad, La Jolla, Calif.) was used. P<0.05 was considered to be significant. Asterisks in graphs denote statistically significant differences.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Nemeth E & Ganz T (2009) The role of hepcidin in iron metabolism. Acta Haematol 122(2-3):78-86.
2. Polishchuk R S & Polishchuk E V (2019) From and to the Golgi—defining the Wilson disease protein road map. FEBS Lett 593(17):2341-2350.
3. Forbes J R & Cox D W (2000) Copper-dependent trafficking of Wilson disease mutant ATP7B proteins. Hum Mol Genet 9(13):1927-1935.
4. Roy S, et al. (2020) Analysis of Wilson disease mutations revealed that interactions between different ATP7B mutants modify their properties. Sci Rep 10(1):13487.
5. Casu C, Nemeth E, & Rivella S (2018) Hepcidin agonists as therapeutic tools. Blood 131(16):1790-1794.
6. Culotta V C, Yang M, & Hall M D (2005) Manganese transport and trafficking: lessons learned from Saccharomyces cerevisiae. Eukaryot Cell 4(7):1159-1165.
7. Jensen L T, et al. (2009) Down-regulation of a manganese transporter in the face of metal toxicity. Mol Biol Cell 20(12):2810-2819.
8. Britton A A & Cotzias G C (1966) Dependence of manganese turnover on intake. Am J Physiol 211(1):203-206.
9. Cotzias G C & Greenough J J (1958) The high specificity of the manganese pathway through the body. J Clin Invest 37(9):1298-1305.
10. Papavasiliou P S, Miller S T, & Cotzias G C (1966) Role of liver in regulating distribution and excretion of manganese. Am J Physiol 211(1):211-216.
12. Racette B A, et al. (2012) Increased risk of parkinsonism associated with welding exposure. Neurotoxicology 33(5):1356-1361.
13. Aschner M, Erikson K M, Herrero Hernandez E, & Tjalkens R (2009) Manganese and its role in Parkinson's disease: from transport to neuropathology. Neuromolecular Med 11(4):252-266.
15. Bouchard M, Laforest F, Vandelac L, Bellinger D, & Mergler D (2007) Hair manganese and hyperactive behaviors: pilot study of school-age children exposed through tap water. Environ Health Perspect 115(1):122-127.
16. Bouchard M F, et al. (2011) Intellectual impairment in school-age children exposed to manganese from drinking water. Environ Health Perspect 119(1):138-143.
17. Claus Henn B, et al. (2010) Early postnatal blood manganese levels and children's neurodevelopment. Epidemiology 21(4):433-439.
18. Khan K, et al. (2011) Manganese exposure from drinking water and children's classroom behavior in Bangladesh. Environ Health Perspect 119(10):1501-1506.
19. Khan K, et al. (2012) Manganese exposure from drinking water and children's academic achievement. Neurotoxicology 33(1):91-97.
20. Lucchini R G, et al. (2012) Tremor, olfactory and motor changes in Italian adolescents exposed to historical ferro-manganese emission. Neurotoxicology 33(4):687-696.
21. Oulhote Y, et al. (2014) Neurobehavioral function in school-age children exposed to manganese in drinking water. Environ Health Perspect 122(12):1343-1350.
22. Riojas-Rodriguez H, et al. (2010) Intellectual function in Mexican children living in a mining area and environmentally exposed to manganese. Environ Health Perspect 118(10):1465-1470.
23. Wasserman G A, et al. (2006) Water manganese exposure and children's intellectual function in Araihazar, Bangladesh. Environ Health Perspect 114(1):124-129.
25. Butterworth R F (2013) Parkinsonism in cirrhosis: pathogenesis and current therapeutic options. Metab Brain Dis 28(2):261-267.
26. Butterworth R F, Spahr L, Fontaine S, & Layrargues G P (1995) Manganese toxicity, dopaminergic dysfunction and hepatic encephalopathy. Metab Brain Dis 10(4):259-267.
28. Spahr L, et al. (1996) Increased blood manganese in cirrhotic patients: relationship to pallidal magnetic resonance signal hyperintensity and neurological symptoms. Hepatology 24(5):1116-1120.
29. Layrargues G P, Shapcott D, Spahr L, & Butterworth R F (1995) Accumulation of manganese and copper in pallidum of cirrhotic patients: role in the pathogenesis of hepatic encephalopathy? Metab Brain Dis 10(4):353-356.
30. Rose C, et al. (1999) Manganese deposition in basal ganglia structures results from both portal-systemic shunting and liver dysfunction. Gastroenterology 117(3):640-644.
31. Burkhard P R, Delavelle J, Du Pasquier R, & Spahr L (2003) Chronic parkinsonism associated with cirrhosis: a distinct subset of acquired hepatocerebral degeneration. Arch Neurol 60(4):521-528.
32. Ballatori N, Miles E, & Clarkson T W (1987) Homeostatic control of manganese excretion in the neonatal rat. Am J Physiol 252(5 Pt 2):R842-847.
33. Bertinchamps A J, Miller S T, & Cotzias G C (1966) Interdependence of routes excreting manganese. Am J Physiol 211(1):217-224.
34. Klaassen C D (1974) Biliary excretion of manganese in rats, rabbits, and dogs. Toxicol Appl Pharmacol 29(3):458-468.
37. Tuschl K, et al. (2012) Syndrome of hepatic cirrhosis, dystonia, polycythemia, and hypermanganesemia caused by mutations in SLC30A10, a manganese transporter in man. Am J Hum Genet 90(3):457-466.
38. Tuschl K, et al. (2008) Hepatic cirrhosis, dystonia, polycythaemia and hypermanganesaemia—a new metabolic disorder. J Inherit Metab Dis 31(2):151-163.
39. Quadri M, et al. (2012) Mutations in SLC30A10 cause parkinsonism and dystonia with hypermanganesemia, polycythemia, and chronic liver disease. Am J Hum Genet 90(3):467-477.
40. Lechpammer M, et al. (2014) Pathology of inherited manganese transporter deficiency. Ann Neurol 75(4):608-612.
42. Perl D P & Olanow C W (2007) The neuropathology of manganese-induced Parkinsonism. J Neuropathol Exp Neurol 66(8):675-682.
44. Gorell J M, et al. (1999) Occupational exposure to manganese, copper, lead, iron, mercury and zinc and the risk of Parkinson's disease. Neurotoxicology 20(2-3):239-247.
45. Boycott K M, et al. (2015) Autosomal-Recessive Intellectual Disability with Cerebellar Atrophy Syndrome Caused by Mutation of the Manganese and Zinc Transporter Gene SLC39A8. Am J Hum Genet 97(6):886-893.
46. Park J H, et al. (2015) SLC39A8 Deficiency: A Disorder of Manganese Transport and Glycosylation. Am J Hum Genet 97(6):894-903.
47. Leyva-Illades D, et al. (2014) SLC30A10 Is a Cell Surface-Localized Manganese Efflux Transporter, and Parkinsonism-Causing Mutations Block Its Intracellular Trafficking and Efflux Activity. J Neurosci 34(42):14079-14095.
48. Zogzas C E, Aschner M, & Mukhopadhyay S (2016) Structural Elements in the Transmembrane and Cytoplasmic Domains of the Metal Transporter SLC30A10 Are Required for Its Manganese Efflux Activity. J Biol Chem 291(31):15940-15957.
49. Zogzas C E & Mukhopadhyay S (2018) Putative metal binding site in the transmembrane domain of the manganese transporter SLC30A10 is different from that of related zinc transporters. Metallomics 10(8):1053-1064.
50. Liu C, et al. (2017) Hypothyroidism induced by loss of the manganese efflux transporter SLC30A10 may be explained by reduced thyroxine production. J Biol Chem 292(40):16605-16615.
51. Taylor C A, et al. (2019) SLC30A10 transporter in the digestive system regulates brain manganese under basal conditions while brain SLC30A10 protects against neurotoxicity. J Biol Chem 294(6):1860-1876.
53. Balachandran R C, et al. (2020) Brain manganese and the balance between essential roles and neurotoxicity. J Biol Chem 295(19):6312-6329.
54. Jenkitkasemwong S, Wang C Y, Mackenzie B, & Knutson M D (2012) Physiologic implications of metal-ion transport by ZIP14 and ZIP8. Biometals 25(4):643-655.
55. Aydemir T B, et al. (2017) Metal Transporter Zip14 (Slc39a14) Deletion in Mice Increases Manganese Deposition and Produces Neurotoxic Signatures and Diminished Motor Activity. J Neurosci 37(25):5996-6006.
56. Jenkitkasemwong S, et al. (2018) SLC39A14 deficiency alters manganese homeostasis and excretion resulting in brain manganese accumulation and motor deficits in mice. Proc Natl Acad Sci USA 115(8):E1769-E1778.
58. Scheiber I F, Wu Y, Morgan S E, & Zhao N (2019) The intestinal metal transporter ZIP14 maintains systemic manganese homeostasis. J Biol Chem 294(23):9147-9160.
59. Aydemir T B, et al. (2020) Intestine-specific deletion of metal transporter Zip14 (Slc39a14) causes brain manganese overload and locomotor defects of manganism. Am J Physiol Gastrointest Liver Physiol 318(4):G673-G681.
60. Lin W, et al. (2017) Hepatic metal ion transporter ZIP8 regulates manganese homeostasis and manganese-dependent enzyme activity. J Clin Invest 127(6):2407-2417.
61. Jeong J & Eide D J (2013) The SLC39 family of zinc transporters. Mol Aspects Med 34(2-3):612-619.
63. Mukhopadhyay S & Linstedt A D (2011) Identification of a gain-of-function mutation in a Golgi P-type ATPase that enhances manganese2+efflux and protects against toxicity. Proc Natl Acad Sci USA 108(2):858-863.
64. Bensaude O (2011) Inhibiting eukaryotic transcription: Which compound to choose? How to evaluate its activity? Transcription 2(3):103-108.
65. Farre D, et al. (2003) Identification of patterns in biological sequences at the ALGGEN server: PROMO and MALGEN. Nucleic Acids Res 31(13):3651-3653.
66. Messeguer X, et al. (2002) PROMO: detection of known transcription regulatory elements using species-tailored searches. Bioinformatics 18(2):333-334.
67. Schummer P, Kuphal S, Vardimon L, Bosserhoff A K, & Kappelmann M (2016) Specific c-Jun target genes in malignant melanoma. Cancer Biol Ther 17(5):486-497.
68. Chen L, et al. (2010) Genome-wide analysis of YY2 versus YY1 target genes. Nucleic Acids Res 38(12):4011-4026.
69. Zhao X, et al. (2019) Mechanisms involved in the activation of C/EBPalpha by small activating RNA in hepatocellular carcinoma. Oncogene 38(18):3446-3457.
70. Kim J, et al. (2019) Wild-Type p53 Promotes Cancer Metabolic Switch by Inducing PUMA-Dependent Suppression of Oxidative Phosphorylation. Cancer Cell 35(2):191-203 e198.
71. Cai S Y, He H, Nguyen T, Mennone A, & Boyer J L (2010) Retinoic acid represses CYP7A1 expression in human hepatocytes and HepG2 cells by FXR/RXR-dependent and independent mechanisms. J Lipid Res 51(8):2265-2274.
72. Majmundar A J, Wong W J, & Simon M C (2010) Hypoxia-inducible factors and the response to hypoxic stress. Mol Cell 40(2):294-309.
73. Mole D R, et al. (2009) Genome-wide association of hypoxia-inducible factor (HIF)-1alpha and HIF-2alpha DNA binding with expression profiling of hypoxia-inducible transcripts. J Biol Chem 284(25):16767-16775.
74. Kaelin W G (2005) Proline hydroxylation and gene expression. Annu Rev Biochem 74:115-128.
75. Hirsila M, Koivunen P, Gunzler V, Kivirikko K I, & Myllyharju J (2003) Characterization of the human prolyl 4-hydroxylases that modify the hypoxia-inducible factor. J Biol Chem 278(33):30772-30780.
76. Li Q, Chen H, Huang X, & Costa M (2006) Effects of 12 metal ions on iron regulatory protein 1 (IRP-1) and hypoxia-inducible factor-1 alpha (HIF-1 alpha) and HIF-regulated genes. Toxicol Appl Pharmacol 213(3):245-255.
77. Salnikow K, et al. (2004) Depletion of intracellular ascorbate by the carcinogenic metals nickel and cobalt results in the induction of hypoxic stress. J Biol Chem 279(39):40337-40344.
78. Davidson T L, Chen H, Di Toro D M, D'Angelo G, & Costa M (2006) Soluble nickel inhibits HIF-prolyl-hydroxylases creating persistent hypoxic signaling in A549 cells. Mol Carcinog 45(7):479-489.
79. Maxwell P & Salnikow K (2004) HIF-1: an oxygen and metal responsive transcription factor. Cancer Biol Ther 3(1):29-35.
80. Han J, et al. (2009) Manganese (II) induces chemical hypoxia by inhibiting HIF-prolyl hydroxylase: implication in manganese-induced pulmonary inflammation. Toxicol Appl Pharmacol 235(3):261-267.
82. Lee K, et al. (2010) LW6, a novel HIF-1 inhibitor, promotes proteasomal degradation of HIF-1alpha via upregulation of VHL in a colon cancer cell line. Biochem Pharmacol 80(7):982-989.
83. Sanghani N S & Haase V H (2019) Hypoxia-Inducible Factor Activators in Renal Anemia: Current Clinical Experience. Adv Chronic Kidney Dis 26(4):253-266.
84. Besarab A, et al. (2016) Roxadustat (FG-4592): Correction of Anemia in Incident Dialysis Patients. J Am Soc Nephrol 27(4):1225-1233.
85. Pergola P E, Spinowitz B S, Hartman C S, Maroni B J, & Haase V H (2016) Vadadustat, a novel oral HIF stabilizer, provides effective anemia treatment in nondialysis-dependent chronic kidney disease. Kidney Int 90(5):1115-1122.
86. Li Z L, Tu Y, & Liu B C (2020) Treatment of Renal Anemia with Roxadustat: Advantages and Achievement. Kidney Dis (Basel) 6(2):65-73.
87. Wade C M & Daly M J (2005) Genetic variation in laboratory mice. Nat Genet 37(11):1175-1180.
88. Lilue J, et al. (2018) Sixteen diverse laboratory mouse reference genomes define strain-specific haplotypes and novel functional loci. Nat Genet 50(11):1574-1583.
89. Yoshiki A & Moriwaki K (2006) Mouse phenome research: implications of genetic background. ILAR J 47(2):94-102.
91. Fleming R E, et al. (2001) Mouse strain differences determine severity of iron accumulation in Hfe knockout model of hereditary hemochromatosis. Proc Natl Acad Sci USA 98(5):2707-2711.
92. Ebert B L & Bunn H F (1999) Regulation of the erythropoietin gene. Blood 94(6):1864-1877.
93. Wahlberg K, et al. (2016) Common Polymorphisms in the Solute Carrier SLC30A10 are Associated With Blood Manganese and Neurological Function. Toxicol Sci 149(2):473-483
95. Ahmad T R, et al. (2020) Bile acid composition regulates the manganese transporter Slc30a10 in intestine. J Biol Chem 295(35):12545-12558.
97. Li S, et al. (2020) Analysis of 1,25-Dihydroxyvitamin D3 Genomic Action Reveals Calcium-Regulating and Calcium-Independent Effects in Mouse Intestine and Human Enteroids. Mol Cell Biol 41(1).
98. Selyunin A S, Iles L R, Bartholomeusz G, & Mukhopadhyay S (2017) Genome-wide siRNA screen identifies UNC50 as a regulator of Shiga toxin 2 trafficking. J Cell Biol 216(10):3249-3262.
99. Hutchens S, et al. (2017) Deficiency in the manganese efflux transporter SLC30A10 induces severe hypothyroidism in mice. J Biol Chem 292(23):9760-9773.
100. Coneyworth L J, et al. (2012) Identification of the human zinc transcriptional regulatory element (ZTRE): a palindromic protein-binding DNA sequence responsible for zinc-induced transcriptional repression. J Biol Chem 287(43):36567-36581.
101. Schulman I G, Juguilon H, & Evans R M (1996) Activation and repression by nuclear hormone receptors: hormone modulates an equilibrium between active and repressive states. Molecular and Cellular Biology 16(7):3807-3813.
102. Qiao H & May J M (2012) Interaction of the transcription start site core region and transcription factor YY1 determine ascorbate transporter SVCT2 exon 1a promoter activity. PloS one 7(4):e35746.
103. Briggs K J, et al. (2016) Paracrine Induction of HIF by Glutamate in Breast Cancer: Eg1N1 Senses Cysteine. Cell 166(1):126-139
104. Wiederschain D, et al. (2009) Single-vector inducible lentiviral RNAi system for oncology target validation. Cell Cycle 8(3):498-504.

What is claimed is:

1. A method for treating manganese (Mn) toxicity in a subject, the method comprising administering to the subject an effective amount of a hypoxia inducible factor (HIF) prolyl hydroxylase inhibitor or a pharmaceutically acceptable salt or solvate thereof.

2. The method of claim 1, wherein the HIF prolyl hydroxylase inhibitor is daprodustat, desidustat, enarodustat, molidustat, roxadustat, vadadustat, or any combinations thereof.

3. The method of claim 1, wherein the HIF prolyl hydroxylase inhibitor is molidustat, roxadustat, vadadustat, or any combinations thereof.

4. The method of claim 1, wherein the HIF prolyl hydroxylase inhibitor is roxadustat.

5. The method of claim 1, wherein the subject has manganese-induced parkinsonism or is at a risk of developing manganese-induced parkinsonism, or cirrhosis.

6. The method of claim 1, wherein the subject has an increased blood manganese concentration when compared to a standard.

7. The method of claim 1, wherein the subject has a blood manganese concentration greater than 7 μg/L.

8. The method of claim 1, wherein the subject has an increased bone manganese concentration when compared to a standard.

9. The method of claim 1, wherein the subject has manganese deposition in liver and/or brain.

10. The method of claim 1, wherein manganese concentration in basal ganglia of the subject is higher, compared to manganese concentration in other areas of the brain of the subject.

11. The method of claim 1, wherein the subject has neuronal degeneration at the basal ganglia, or is at a risk of developing neuronal degeneration at the basal ganglia.

12. The method of claim 1, wherein the subject has homozygous loss of function mutations in the SLC30A10 gene and/or the SLC39A14 gene.

13. The method of claim 1, wherein the subject has single nucleotide polymorphism in the SLC30A10 gene.

14. The method of claim 1, wherein the subject has SLC39A14 deficiency in the brain, liver, and/or intestine.

15. The method of claim 1, wherein the subject has increased expression of SLC30A10 in the brain, liver, and/or intestine.

16. The method of claim 1, wherein the HIF prolyl hydroxylase inhibitor or a pharmaceutically acceptable salt or solvate thereof is administered orally, intravenously, parenterally, subcutaneously, or intramuscularly.

17. A method for treating manganese-induced parkinsonism in a subject, the method comprises administering to the subject an effective amount of a hypoxia inducible factor (HIF) prolyl hydroxylase inhibitor, or a pharmaceutically acceptable salt or solvate thereof.

18. The method of claim 17, wherein the HIF prolyl hydroxylase inhibitor is daprodustat, desidustat, enarodustat, molidustat, roxadustat, vadadustat, or any combinations thereof.

19. A method for treating manganese-induced neurotoxicity in a subject, the method comprises administering to the subject an effective amount of a hypoxia inducible factor (HIF) prolyl hydroxylase inhibitor, or a pharmaceutically acceptable salt or solvate thereof.

20. The method of claim 19, wherein the HIF prolyl hydroxylase inhibitor is daprodustat, desidustat, enarodustat, molidustat, roxadustat, vadadustat, or any combinations thereof.


SEQUENCE LISTING (52 sequences)

SEQ ID NO: 1 - DNA, 21 bp, synthetic construct
  source 1..21: mol_type = other DNA
tgctctttgtggttggatcta

SEQ ID NO: 2 - DNA, 21 bp, synthetic construct
  source 1..21: mol_type = other DNA
cagtacccagacggatttcaa

SEQ ID NO: 3 - DNA, 21 bp, synthetic construct
  source 1..21: mol_type = other DNA
agcctcatcatcgttcaagtt

SEQ ID NO: 4 - DNA, 21 bp, synthetic construct
  source 1..21: mol_type = other DNA
gcctacactctccaacacaat

SEQ ID NO: 5 - DNA, 21 bp, synthetic construct
  source 1..21: mol_type = other DNA
caacaagatgaagagcaccaa

SEQ ID NO: 6 - DNA, 21 bp, synthetic construct
  source 1..21: mol_type = other DNA
gtggactcttgaaagtactat

SEQ ID NO: 7 - DNA, 20 bp, synthetic construct
  source 1..20: mol_type = other DNA
ccacaggacagtacaggatg

SEQ ID NO: 8 - DNA, 22 bp, synthetic construct
  source 1..22: mol_type = other DNA
tcaagtcgtgctgaataatacc

SEQ ID NO: 9 - DNA, 20 bp, synthetic construct
  source 1..20: mol_type = other DNA
gcgacaatgacagctgacaa

SEQ ID NO: 10 - DNA, 18 bp, synthetic construct
  source 1..18: mol_type = other DNA
cagcatcccgggacttct

SEQ ID NO: 11 - DNA, 20 bp, synthetic construct
  source 1..20: mol_type = other DNA
ctagtggccattggcagatt

SEQ ID NO: 12 - DNA, 20 bp, synthetic construct
  source 1..20: mol_type = other DNA
caatgttgtgtcgggagatg

SEQ ID NO: 13 - DNA, 20 bp, synthetic construct
  source 1..20: mol_type = other DNA
atgctacccaaataaccagc

SEQ ID NO: 14 - DNA, 21 bp, synthetic construct
  source 1..21: mol_type = other DNA
caggaatccatatccccaaac

SEQ ID NO: 15 - DNA, 20 bp, synthetic construct
  source 1..20: mol_type = other DNA
tggcttatctgggctttgtg

SEQ ID NO: 16 - DNA, 20 bp, synthetic construct
  source 1..20: mol_type = other DNA
cacactggctctgatggcta

SEQ ID NO: 17 - DNA, 20 bp, synthetic construct
  source 1..20: mol_type = other DNA
tgtctccaagtctgcagtgg

SEQ ID NO: 18 - DNA, 20 bp, synthetic construct
  source 1..20: mol_type = other DNA
ggaatcatgtggtccaggtc

SEQ ID NO: 19 - DNA, 21 bp, synthetic construct
  source 1..21: mol_type = other DNA
ttcccgcttatcaaggagacc

SEQ ID NO: 20 - DNA, 21 bp, synthetic construct
  source 1..21: mol_type = other DNA
actgctaattccaggcacagc

SEQ ID NO: 21 - DNA, 20 bp, synthetic construct
  source 1..20: mol_type = other DNA
cagttaaccaacatcttagc

SEQ ID NO: 22 - DNA, 19 bp, synthetic construct
  source 1..19: mol_type = other DNA
aagctcatggatgttagag

SEQ ID NO: 23 - DNA, 23 bp, synthetic construct
  source 1..23: mol_type = other DNA
ggatagagttcctgctgacttac

SEQ ID NO: 24 - DNA, 20 bp, synthetic construct
  source 1..20: mol_type = other DNA
tgaggagctgtcaccttaga

SEQ ID NO: 25 - DNA, 19 bp, synthetic construct
  source 1..19: mol_type = other DNA
gggtctcgattggatggca

SEQ ID NO: 26 - DNA, 21 bp, synthetic construct
  source 1..21: mol_type = other DNA
agggcagaatcatcacgaagt

SEQ ID NO: 27 - DNA, 19 bp, synthetic construct
  source 1..19: mol_type = other DNA
ccgccgcctttttgggtag

SEQ ID NO: 28 - DNA, 20 bp, synthetic construct
  source 1..20: mol_type = other DNA
gagtcagggaccttcagcag

SEQ ID NO: 29 - DNA, 19 bp, synthetic construct
  source 1..19: mol_type = other DNA
cgcctatgggaaggtgttc

SEQ ID NO: 30 - DNA, 19 bp, synthetic construct
  source 1..19: mol_type = other DNA
ttggggtgctcgaaggtct

SEQ ID NO: 31 - DNA, 21 bp, synthetic construct
  source 1..21: mol_type = other DNA
acctggagactctcagggtcg

SEQ ID NO: 32 - DNA, 24 bp, synthetic construct
  source 1..24: mol_type = other DNA
ttagggcttcctcttggagaagat

SEQ ID NO: 33 - DNA, 20 bp, synthetic construct
  source 1..20: mol_type = other DNA
ctgtgaatcctgtgctctgc

SEQ ID NO: 34 - DNA, 20 bp, synthetic construct
  source 1..20: mol_type = other DNA
aatgaatgccagtggtcaca

SEQ ID NO: 35 - DNA, 20 bp, synthetic construct
  source 1..20: mol_type = other DNA
ccataaggtgttgtgccacg

SEQ ID NO: 36 - DNA, 20 bp, synthetic construct
  source 1..20: mol_type = other DNA
catccatcgggtcaatgctt

SEQ ID NO: 37 - DNA, 21 bp, synthetic construct
  source 1..21: mol_type = other DNA
cgaaccacggcactgattttc

SEQ ID NO: 38 - DNA, 21 bp, synthetic construct
  source 1..21: mol_type = other DNA
tttcttgctgccagtctggac

SEQ ID NO: 39 - DNA, 18 bp, synthetic construct
  source 1..18: mol_type = other DNA
aagtccctgctcgaccac

SEQ ID NO: 40 - DNA, 18 bp, synthetic construct
  source 1..18: mol_type = other DNA
ctgggaatccagctgctg

SEQ ID NO: 41 - DNA, 20 bp, synthetic construct
  source 1..20: mol_type = other DNA
ctcgccttcagtgaggatgt

SEQ ID NO: 42 - DNA, 20 bp, synthetic construct
  source 1..20: mol_type = other DNA
gctttgcgttgtgctttctt

SEQ ID NO: 43 - DNA, 20 bp, synthetic construct
  source 1..20: mol_type = other DNA
tcagagctccaccatgactg

SEQ ID NO: 44 - DNA, 20 bp, synthetic construct
  source 1..20: mol_type = other DNA
tgtgaacgtgaggatgggta

SEQ ID NO: 45 - DNA, 21 bp, synthetic construct
  source 1..21: mol_type = other DNA
gtagcaggtgattccctgaac

SEQ ID NO: 46 - DNA, 21 bp, synthetic construct
  source 1..21: mol_type = other DNA
gtgatgaccacaaccacggac

SEQ ID NO: 47 - DNA, 21 bp, synthetic construct
  source 1..21: mol_type = other DNA
ttgcaggagtcattgctgcta

SEQ ID NO: 48 - DNA, 22 bp, synthetic construct
  source 1..22: mol_type = other DNA
tggagttctgcacaccattgat

SEQ ID NO: 49 - DNA, 22 bp, synthetic construct
  source 1..22: mol_type = other DNA
gactctagcccttggtgttatg

SEQ ID NO: 50 - DNA, 20 bp, synthetic construct
  source 1..20: mol_type = other DNA
cttcgtcagggttccagttt

SEQ ID NO: 51 - DNA, 19 bp, synthetic construct
  source 1..19: mol_type = other DNA
cattaagggcgtggggcgg

SEQ ID NO: 52 - DNA, 20 bp, synthetic construct
  source 1..20: mol_type = other DNA
gtcgtgggttctgcatgatg